(19) United States
(12) Patent Application Publication
(10) Pub. No.: US 2019/0059875 A1
(43) Pub. Date: Feb. 28, 2019

(54) CANNULATED ANCHOR SYSTEM AND METHOD

(71) Applicant: TIGON MEDICAL, Millersville, MD (US)

(72) Inventors: Umasuthan SRIKUMARAN, Ellicott City, MD (US); Jeremy CLARK, Millersville, MD (US)

(21) Appl. No.: 16/106,678

(22) Filed: Aug. 21, 2018

Related U.S. Application Data
(60) Provisional application No. 62/548,781, filed on Aug. 22, 2017.

(51) Int. Cl. A61B 17/04

(52) U.S. Cl. CPC A61B 17/0401; A61B 2017/0445; A61B 2017/044; A61B 2017/0409

(57) ABSTRACT

A cannulated suture anchor may include a threaded portion and a tip portion. The threaded portion may include a continuous thread from a proximal end to a distal end and a suture opening located in the body of the threaded portion. The tip portion may be provided at the distal end of the threaded portion and may include a frustoconical, non-threaded outer surface. The cannulated suture anchor may be inserted into bone with an awl in a single step. Methods of using the cannulated suture anchor are also provided.

### CROSS-REFERENCE TO RELATED APPLICATIONS

This application claims the benefit of U.S. Provisional Patent Application Ser. No. 62/548,781 entitled “CANNULATED ANCHOR SYSTEM AND METHOD,” filed Aug. 22, 2017, the disclosure of which is incorporated by reference herein in its entirety.

### TECHNICAL FIELD

The present application relates to anchors. More specifically, the present application relates to anchors used in medical procedures, such as rotator cuff repair.

### BACKGROUND OF THE INVENTION

Current suture anchors used in medical procedures, such as rotator cuff repair, are typically constructed of titanium or polyether ether ketone (PEEK). Titanium anchors may not require an awl, but an MRI may be scattered due to metal interference. Many medical professionals therefore use PEEK anchors as it is radiolucent. However, PEEK anchors require an awl to make a hole for insertion of the anchor. This is typically a two-step process, the first step for insertion of the awl to create an initial hole and the second step for installing the anchor in the bone. Current anchors typically do not allow for more than one suture tape to be attached in a single anchor. A suture tape is a large flat suture which is used to hold the rotator cuff down to the bone for healing and is typically larger in size than a suture. A suture tape may be larger than a suture, and thus, due to its size, current anchors are not capable of supporting more than one of the larger suture tapes. Current anchors may be provided as cannulated (hollow) anchors or solid anchors. Solid anchors that may be strong enough to insert directly into the bone do not allow for the flow of biological healing products, such as bone marrow elements, from accessing the repair site. Thus, a need exists for a cannulated anchor which may support two or more suture tapes, may be inserted into a patient's bone in a single step, and which allows venting of biologic elements directly to the bone tendon interface where healing is needed.

### BRIEF SUMMARY OF THE INVENTION

According to an embodiment, a suture anchor may include a threaded portion, the threaded portion including: a body, a suture opening in a side of the body, the opening configured to receive a suture tape, a continuous thread on an outer surface of the body, and a bore extending from a body proximal surface to a body distal surface. The suture anchor may further include a tip portion, the tip portion including an outer surface without the continuous thread and a bore extending from a tip portion proximal surface to a tip portion distal surface, the tip portion being tapered from a tapered portion proximal surface to the tip portion distal surface. The bore of the threaded portion aligns with the bore of the tip portion, thereby creating a continuous bore from the body proximal surface to the tip portion distal surface such that the suture anchor is open at the body proximal surface and open at the tip portion distal surface. The outer surface of the tip portion is smooth. The suture opening is located at a proximal end of the threaded portion. The continuous thread includes a crest, a flank, and a root, and wherein the opening extends through at least one of the crest, the flank, and the root. The bore of the threaded portion further comprising a first bore, a second bore, and a third bore, and wherein the suture opening connects to the first bore. The first bore is located at a proximal end of the threaded portion. The anchor may include a vent opening in the body, the vent opening configured to allow flow of fluids through the suture anchor. A diameter of the third bore is substantially the same as a diameter of the bore of the tip portion. The tip portion further comprises a non-tapered portion extending from the tip portion proximal surface to the tapered portion proximal surface and a tapered portion extending from the tapered portion proximal surface to the tip portion distal surface. The tapered portion proximal surface is configured to mate with the body distal surface. The continuous thread extends from the body proximal surface to the body distal surface. The suture anchor comprises polyether ether ketone. The tip portion comprises one of polyether ether ketone and metal. The tip portion is frustoconical. The anchor may include one or more ventilation openings. The threaded portion and the tip portion are integral.

According to an embodiment, a cannulated anchor system, may include a suture anchor, the suture anchor including: a body, the body including an outer surface and an inner surface defining a longitudinal bore, a suture opening in a side of the body, the suture opening configured to receive one or more suture tapes and the opening located at a proximal end of the body, and an awl, the awl including: a handle, and a medial shaft attached to the handle, the medial shaft having a pointed tip. The medial shaft is configured to extend through the longitudinal bore of the body of the suture anchor with the pointed tip extending past a distal end of the suture anchor, and wherein the cannulated anchor system is configured to allow one-step insertion of the suture anchor into a bone. The body of the suture anchor further comprises a threaded portion and a tip portion, the tip portion comprising a smooth outer surface. The threaded portion includes a continuous thread from a proximal surface of the thread portion to a distal surface of the threaded portion, and wherein a proximal surface of the tip portion is configured to mate with the distal surface of the threaded portion. The anchor system may include an annular space between the medial shaft and the inner surface of the body, wherein the annular space is configured to allow passage of the one or more suture tapes. The one or more suture tapes extend into the annular space and through the opening to and along the outer surface. The medial shaft comprises a first rod and a second rod, and a substantially ellipsoidal portion located between the first rod and the second rod. The first rod comprises a first diameter portion and a second diameter portion smaller than the first diameter portion, and the second diameter portion includes a cutout. The one or more suture tapes comprises two suture tapes. The anchor system may include a cutout in the medial shaft, the cutout configured to allow passage of the one or more suture tapes. The anchor system may include a vent opening in the body of the suture anchor. The anchor system may include a first annular space configured to allow passage of a suture tape from the suture opening through a first opening on a proximal surface of the suture anchor and past a cutout on the medial shaft, and a second annular space configured to allow passage of fluids from a vent opening in the body of the suture anchor to the first opening on the proximal surface of the suture anchor. The pointed tip of the medial shaft extends through a second opening on a distal surface of the suture anchor.

According to an embodiment, a method of installing a suture tape in a bone in a single step, the method may include providing a cannulated anchor having a threaded outer surface, a longitudinal bore, and a first opening extending through the threaded outer surface and located at a proximal end of the cannulated anchor, passing a first suture tape along the threaded outer surface, through the first opening of the cannulated anchor, and through the longitudinal bore, inserting a medial shaft of an awl through the longitudinal bore such that a pointed tip of the medial shaft extends past a distal end of the cannulated anchor and such that the first suture tape is located in an annular space between the medial shaft and an interior surface of the cannulated anchor, punching a hole in the bone with the pointed tip, impacting the bone with a tip portion of the cannulated anchor, enlarging the hole by turning the awl such that the threaded outer surface of the cannulated anchor advances into and enlarges the hole, removing the awl from the cannulated anchor, and securing the first suture tape to a tissue adjacent the bone. The method may include threading a second suture tape through a second opening and securing the second suture tape to the tissue. The method may include reinserting the medial shaft in a second cannulated anchor for installing into another location in the bone.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 shows a perspective view of an anchor, according to an embodiment;

FIG. 2A shows a side view of the anchor of FIG. 1, according to an embodiment;

FIG. 2B shows a top view of the anchor of FIG. 1, according to an embodiment;

FIG. 2C shows an end view of the anchor of FIG. 1, according to an embodiment;

FIG. 3 shows a side cross-sectional view of the anchor of FIG. 1, according to an embodiment;

FIG. 4 shows a perspective view of an anchor, according to an embodiment;

FIG. 5 shows a side view of the anchor of FIG. 4, according to an embodiment;

FIG. 6A shows a perspective view of an anchor, according to an embodiment;

FIG. 6B shows a side cross-sectional view of the anchor of FIG. 6A, according to an embodiment;

FIG. 6C shows a perspective view of a cross-sectional view of the anchor of FIG. 6A, according to an embodiment;

FIG. 6D shows a side cross-sectional view of the anchor of FIG. 6A, according to an embodiment;

FIG. 7A shows a perspective view of a tip portion of an anchor, according to an embodiment;

FIG. 7B shows a side view of the tip portion of FIG. 7A, according to an embodiment;

FIG. 7C shows an end view of the tip portion of FIG. 7A, according to an embodiment;

FIG. 8A shows a side view of a threaded portion of an anchor, according to an embodiment;

FIG. 8B shows a partial cross-section of the threaded portion of FIG. 8A, according to an embodiment;

FIG. 8C shows an end view of the threaded portion of the anchor, according to an embodiment;

FIG. 9 shows a perspective view of an awl, according to an embodiment;

FIG. 10A shows a top view of the awl of FIG. 9, according to an embodiment;

FIG. 10B shows an end view of the awl of FIG. 9, according to an embodiment;

FIG. 11 shows an exploded side view of the awl of FIG. 9, according to an embodiment;

FIG. 12 shows a handle of the awl of FIG. 9, according to an embodiment;

FIG. 13A shows a side view of the handle of FIG. 12, according to an embodiment;

FIG. 13B shows a top view of the handle of FIG. 12, according to an embodiment;

FIG. 13C shows a partial detail view of the handle of FIG. 12, according to an embodiment;

FIG. 13D shows an end view of the handle of FIG. 12, according to an embodiment;

FIG. 14A shows a perspective view of a handle pin of the awl of FIG. 9, according to an embodiment;

FIG. 14B shows a side view of the handle pin of FIG. 14A, according to an embodiment;

FIG. 14C shows an end view of the handle pin of FIG. 14A, according to an embodiment;

FIG. 15A shows a side view of the medial shaft of FIG. 9, according to an embodiment;

FIG. 15B shows a top view of the medial shaft of FIG. 9, according to an embodiment;

FIG. 16A shows a detail view of the medial shaft of FIG. 15A, according to an embodiment;

FIG. 16B shows a detail view of the medial shaft of FIG. 15B, according to an embodiment;

FIG. 16C shows an end view of the medial shaft of FIG. 9, according to an embodiment;

FIG. 17 shows a partial perspective view of the medial shaft of FIG. 9, according to an embodiment;

FIG. 18 shows a partial perspective view of a cannulated anchor system, according to an embodiment;

FIG. 19 shows a cross-sectional view of the cannulated anchor system of FIG. 18, according to an embodiment;

FIG. 20A shows a partial cross-sectional view of the cannulated anchor system of FIG. 18 with a suture tape installed, according to an embodiment;

FIG. 20B shows a partial cross-sectional view of the cannulated anchor system of FIG. 18 with a suture tape installed, with the anchor rotated 90 degrees;

FIG. 21 shows a perspective view of an anchor with a suture tape installed, according to an embodiment;

FIG. 22 shows a perspective view of a tool with a suture tape installed, according to an embodiment;

FIG. 23A shows a side view of a medial shaft, according to an embodiment;

FIG. 23B shows a top view of the medial shaft of FIG. 23A, according to an embodiment;

FIG. 24A shows a detail view of the medial shaft of FIG. 23A, according to an embodiment;

FIG. 24B shows a detail view of the medial shaft of FIG. 23B, according to an embodiment;

FIG. 24C shows an end view of the medial shaft of FIG. 23A, according to an embodiment;

FIG. 25A shows a side view of a handle, according to an embodiment;

FIG. 25B shows a top view of the handle of FIG. 25A, according to an embodiment;

FIGS. 26A through 26D depict various views of another exemplary embodiment of an anchor;

FIGS. 27A through 27E depict various views of another exemplary embodiment of a surgical handle and driving tool;

FIGS. 28A, 28B, 28C and 28D depict various views of a modular awl for use with the various anchor embodiments described herein;

FIGS. 29 and 30 depict an anchor assembled onto the modular awl of FIG. 28A; and

FIGS. 31A and 31B depict views of one exemplary embodiment of an anchor dock and holder for use with the various anchor embodiments disclosed herein.

### DETAILED DESCRIPTION OF THE INVENTION

Embodiments of the invention are discussed in detail below. In describing embodiments, specific terminology is employed for the sake of clarity. However, the inventions are not intended to be limited to the specific terminology so selected. A person skilled in the relevant art would recognize that other equivalent parts can be employed and other methods developed without departing from the spirit and scope of the invention. All references cited herein are incorporated by reference as if each had been individually incorporated.

Various implementations of a cannulated anchor in accordance with the principles of the invention are described herein. The cannulated anchors may support two or more suture tapes, may be inserted into a patient's bone in a single step, and may allow venting of biologic elements directly to the bone tendon interface for healing.

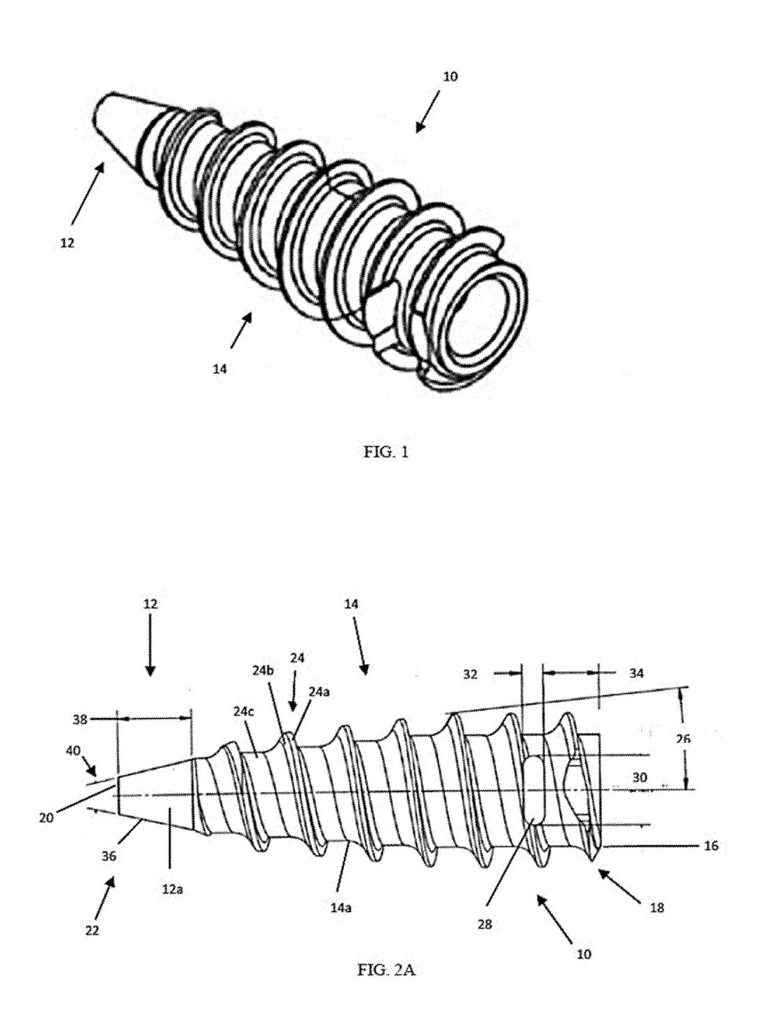

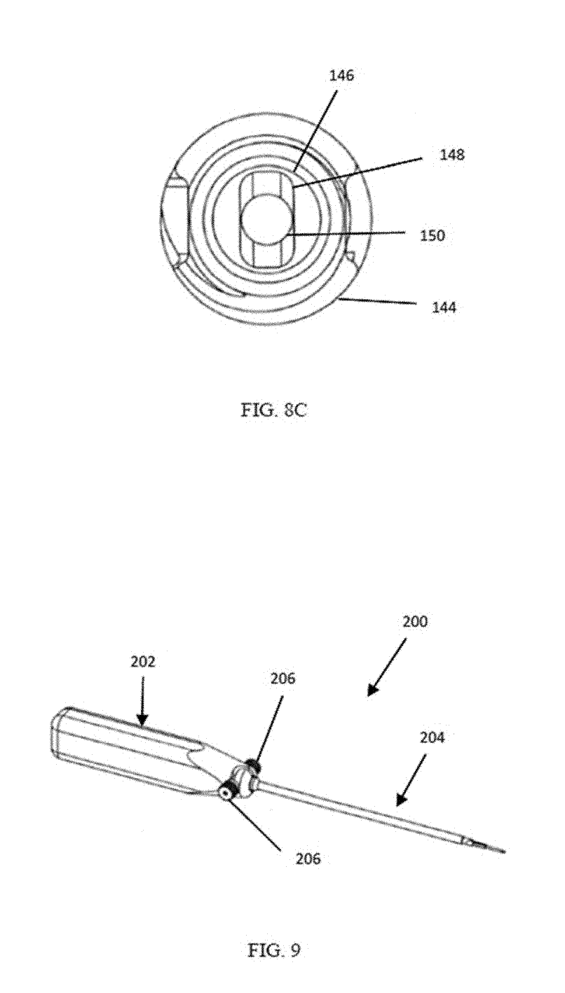

Referring to FIGS. 1-3, views of an anchor 10 are shown. From the following description, it may be appreciated that the anchor 10 generally includes one or more suture openings 28 for accommodating sutures, more specifically suture tapes. The anchor 10 may also include a multi-dimensional cannula to accommodate an awl 200 (FIG. 9). A tip portion 12 of the anchor 10 may be sharp such as to facilitate entry of the anchor 10 into bone during a medical procedure. Referring first to FIG. 1, a perspective view of the anchor 10 is shown. The anchor 10 may include a threaded portion 14 and a tip portion 12. The threaded portion 14 and tip portion 12 may be integral such that anchor 10 is a single piece. The threaded portion 14 and the tip portion 12 may be separate, such that anchor 10 comprises two pieces. The anchor 10 may comprise any material, for example, polyether ether ketone (PEEK). The threaded portion 14 and the tip portion 12 may comprise the same or difference materials. For example, the threaded portion 14 and tip portion 12 may comprise PEEK. Alternatively, the threaded portion 14 may be comprise PEEK and the tip portion 12 may comprise metal. The metal may be any metal known to one skilled in the art. The material may be selected based on the strength of the bone or tissue into which the anchor is being inserted.

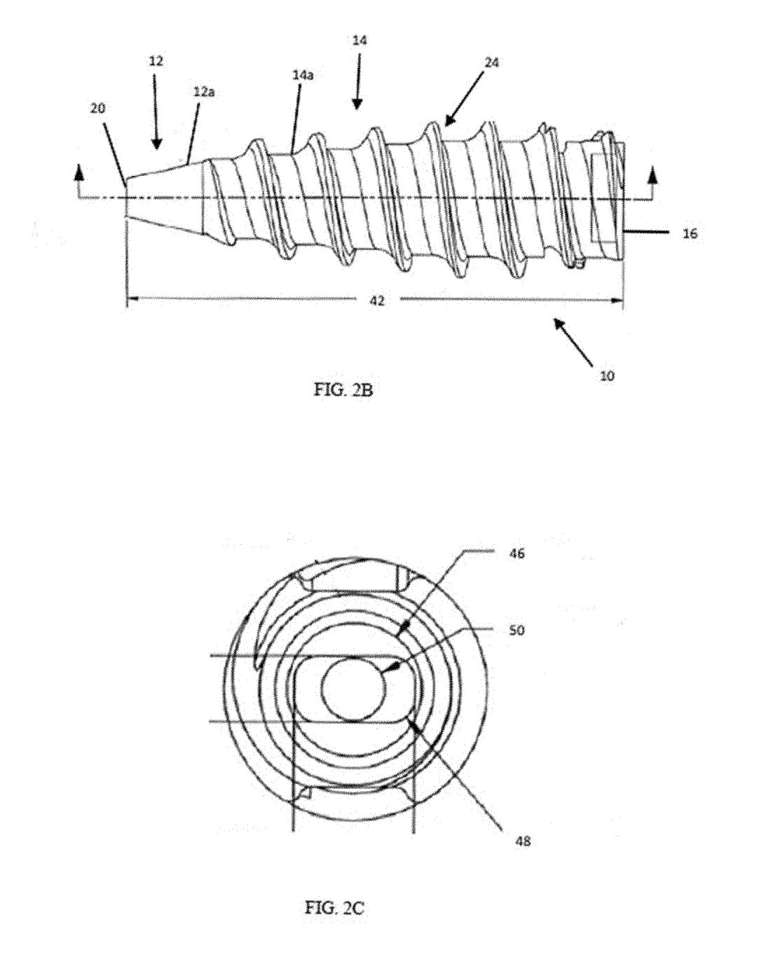

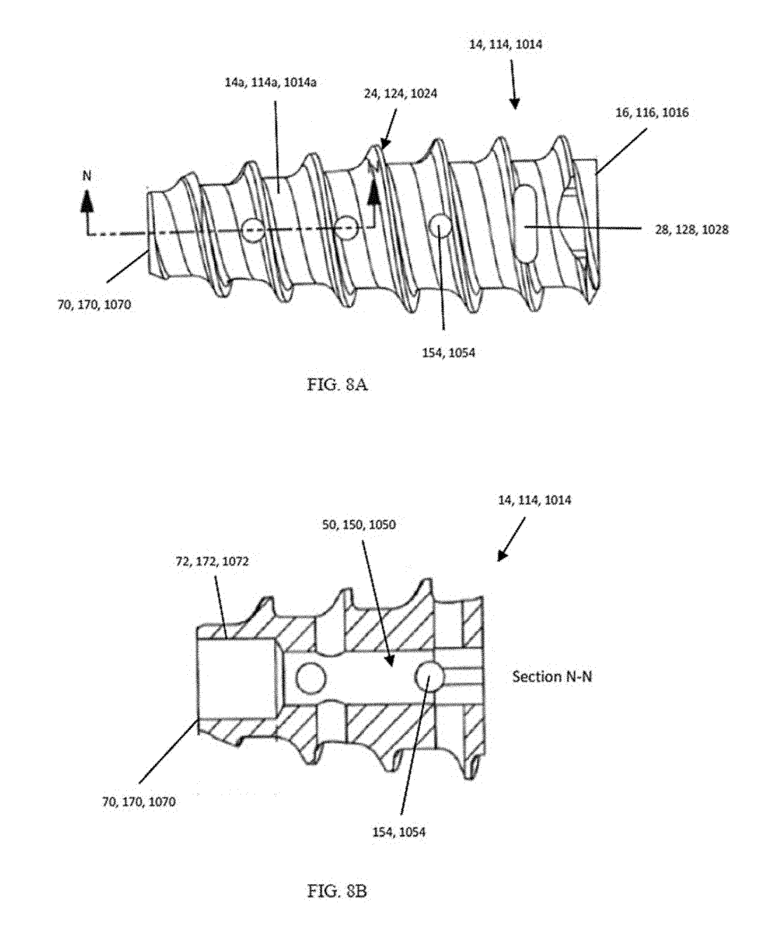

With reference to FIG. 2A, a side view of the anchor 10 is shown. As previously described, the anchor may comprise the threaded portion 14 and the tip portion 12. The anchor 10 may include a proximal surface 16 located a proximal end 18 and a distal surface 20 located at a distal end 22. The anchor 10 may be cannulated, that is, the anchor 10 may have a bore (not shown) which extends from the proximal surface 16 to the distal surface 20. The threaded portion 14 may include a threaded portion body 14a having a thread 24. The thread 24 may comprise a crest 24a, flank 24b, and root 24c. The thread 24 may comprise a thread angle 26 (which may alternative include one or more thread angles, where multiple and/or compound threads are used). The thread angle 26 may be selected to facilitate insertion of the anchor 10 into bone or tissue. The thread 24 may extend continuously from the proximal surface 16 to a threaded portion distal surface 70 (FIG. 8A) of the threaded portion body 14a. The threaded portion body 14a may comprise PEEK, metal, a combination thereof, or other materials.

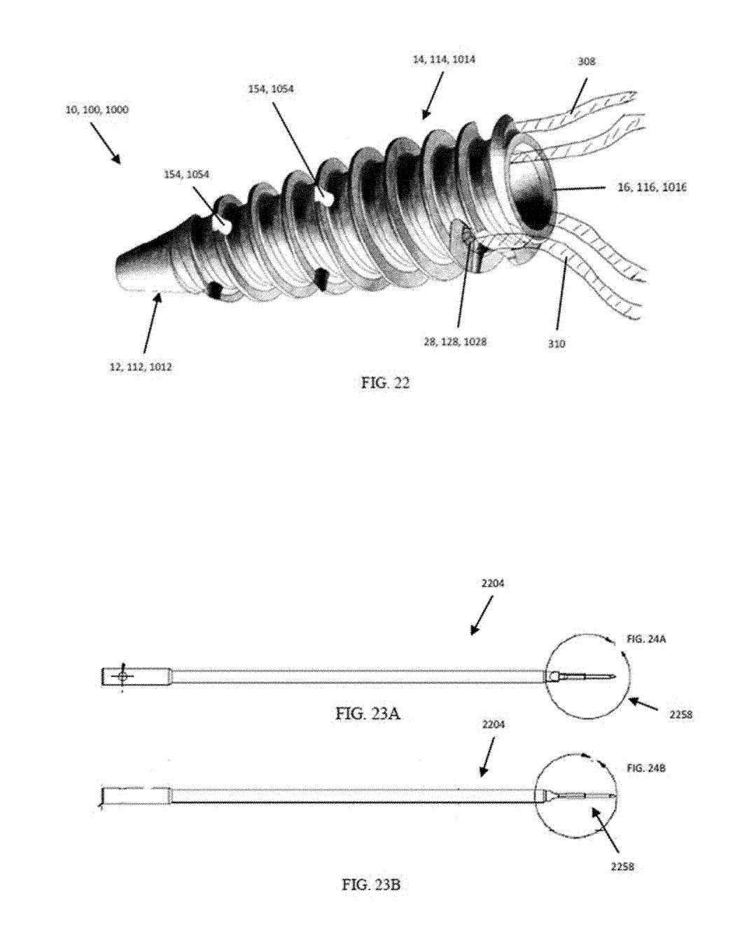

With continued reference to FIG. 2A, the threaded portion 14 may include a suture opening 28. Suture opening 28 may allow for insertion of a suture, tape, or thread to extend from the bore of the threaded portion body 14a, through the suture opening 28, and to the outer surface of the anchor 10. Suture opening 28 may accommodate one or more sutures, tapes, or threads. The suture opening 28 may comprise a length 30 and a width 32. The length 30 and width 32 may be selected to accommodate a suture, tape, or thread of desired size. The length 30 and width 32 may be selected to accommodate one or more sutures, tapes, or threads. The suture opening 28 may be located near the proximal end 18 of the anchor 10. The suture opening 28 may be located a distance 34 from the proximal surface 16 of anchor 10. Although a first suture opening 28 is depicted, it is understood that a second suture opening 28 may be located diametrically opposed to the first suture opening 28, see for example, FIG. 21. Any number of suture openings 28 may be provided in any number of locations about the axis of anchor 10. The suture opening 28 may be located in the thread 24 of the threaded portion 14. The suture opening 28 may be located through the crest 24a, flank 24b, root 24c, or any combination thereof of the thread 24.

In an exemplary embodiment of threaded portion 14, the thread angle 26 may be 6 degrees or 8 degrees. In an exemplary embodiment, the suture opening 28 may have a length 30 of 2.5 mm and a width 32 of 0.75 mm. In an exemplary embodiment, the distance 34 may be 2.00 mm. In an exemplary embodiment, the thread 24 may have a pitch of 1.5 mm or 2.0 mm. It will be understood that the aforementioned dimensions are exemplary and any number of dimensions may be provided.

In various embodiments, the suture opening 28 is desirably positioned near the proximal end of the anchor, which is a somewhat more proximal location than suture openings of various prior art designs. Desirably, this arrangement will allow the surgeon to more easily load suture into and through the opening 28, which in various embodiments can be accomplished without use of a suture passer or similar accessory instrument. If desired, the opening 28 can be formed in an oval and/or oblate spheroid shape, which desirably allows room for multiple suture tapes to be loaded and/or “stacked” into a single opening without compressing, twisting, wrinkling, stretching and/or otherwise compromising the tape. In at least one exemplary embodiment, a proximal inner surface 554a of the opening can incorporate at least one flattened and/or less curved inwardly-facing section, which desirably facilitates the accommodate of a plurality of surgical tapes by the opening.

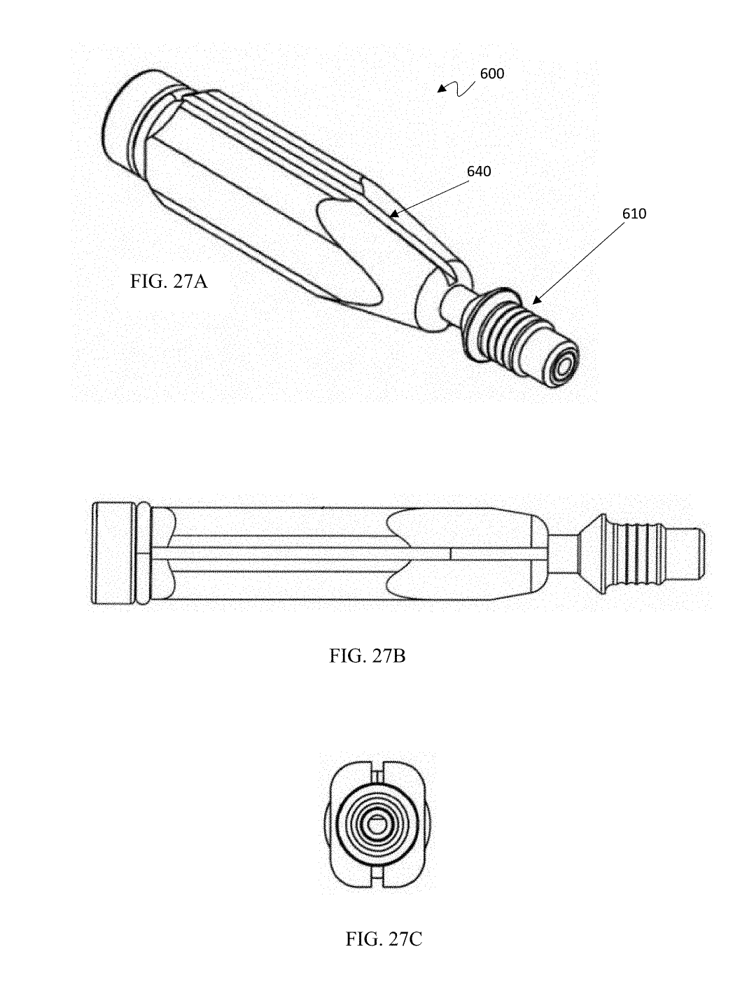

In various embodiments, one or more sections or rivets 527a, 529a (see FIGS. 26A and 26B) on the anchor body can optionally be removed and/or flattened, such as to accommodate thicker and/or more numerous suture tapes and/or to allow the surgical insertion tool to more easily slide into the anchor and suture construct, including the removal and/or flattening of rivets and/or other sections on the outer and/or inner surfaces of the anchor body.

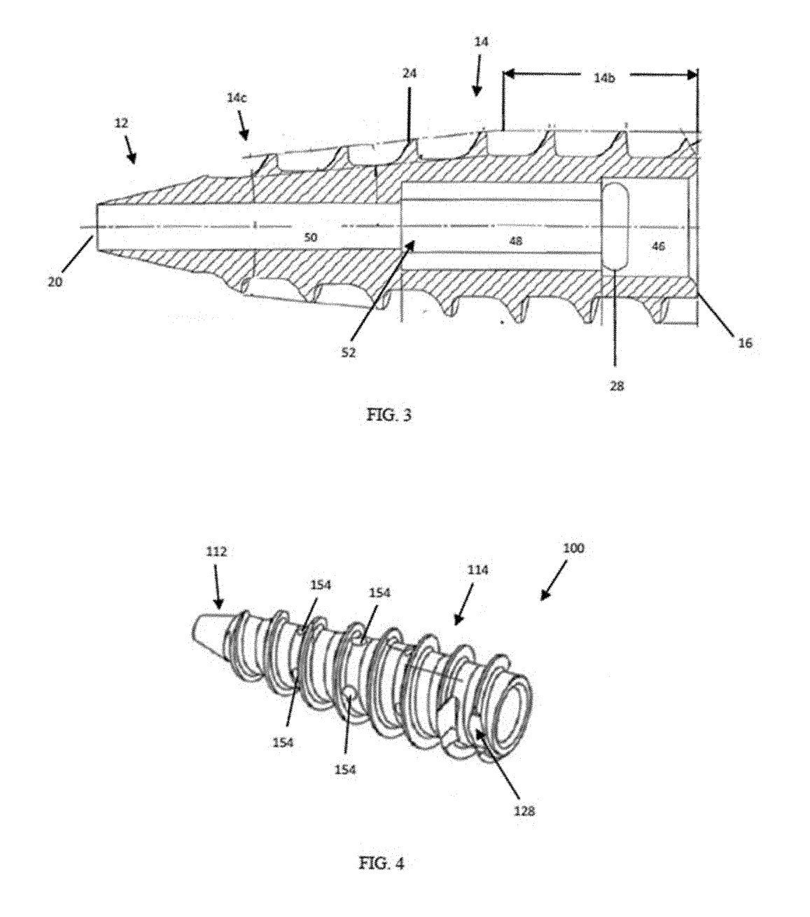

Referring again to FIG. 2A, the tip portion 12 may include a tip body 12a. The tip portion 12 may be tapered such that an end which mates with threaded portion 14 has a greater dimension than the distal surface 20. The tip portion 12 may have a surface 36. The surface 36 may be smooth or flat. The surface 36 may be non-threaded and/or have no protrusions, recesses, or other surface features present. The surface 36 may be smooth, flat, or non-threaded around an entire circumference of tip portion 12. The tip body 12a may comprise PEEK, metal, a combination thereof, or other materials. The distal surface 20 of the tip body 12a may be sharp, that is, the distal surface 20 of the tip body 12a may provide an edge which is able to cut, pierce, or otherwise enter into the bone. As shown, for example, in FIG. 3, the tip portion 12 may taper along the length to form a sharp leading edge at distal surface 20. The sharp leading edge may be configured to cut bone. The tip portion 12 may have a length 38 and a taper angle 40. In an exemplary embodiment of tip portion 12, the length 38 may be 2.63 mm and the taper angle 40 may be 25.7 degrees. The taper angle 40 along with the diameter of the distal surface of the tip body 12a are exemplary dimensions to create the sharp leading edge that is distal surface 20. It will be understood that the aforementioned dimensions are exemplary and any number of dimensions may be provided.

Referring to FIG. 2B, a side view of the anchor 10 can be seen rotated 90 degrees. In this view, it may be seen that the thread 24 may be continuous around the entire circumference of the threaded portion 14. The thread 24 may be continuous from the proximal surface 16 to a threaded portion distal surface 70 (FIG. 8A) of the threaded portion 14. The anchor 10, including the threaded portion 14 and tip portion 12, may have a length 42. In an exemplary embodiment, the length may be 17.13 mm, although other dimensions are contemplated.

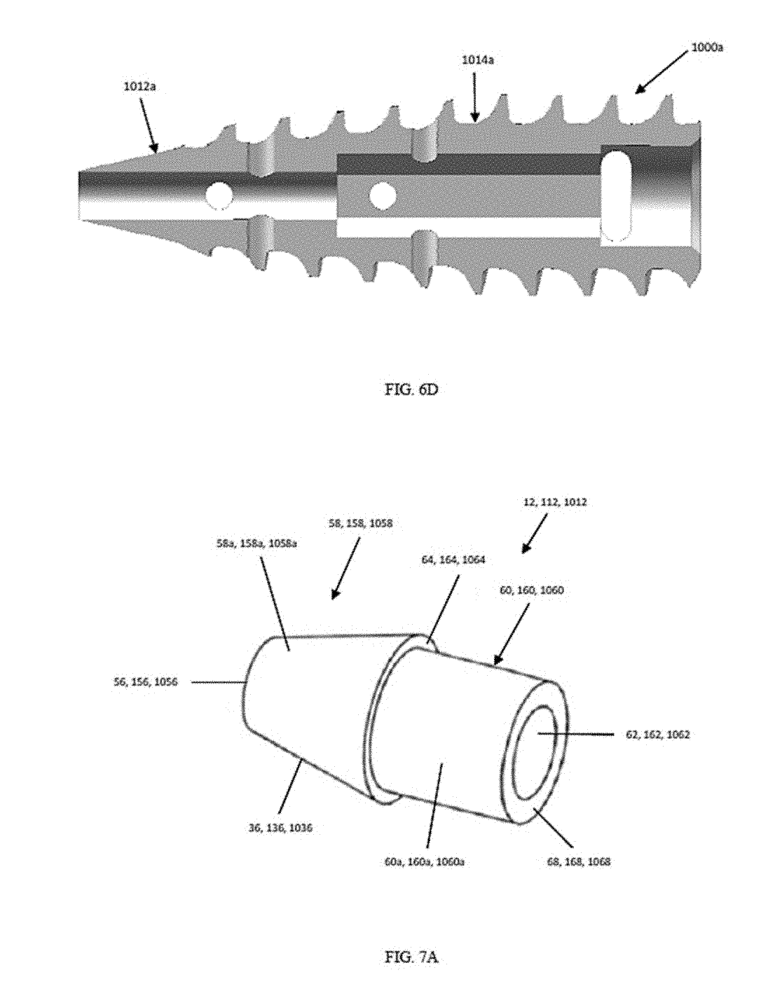

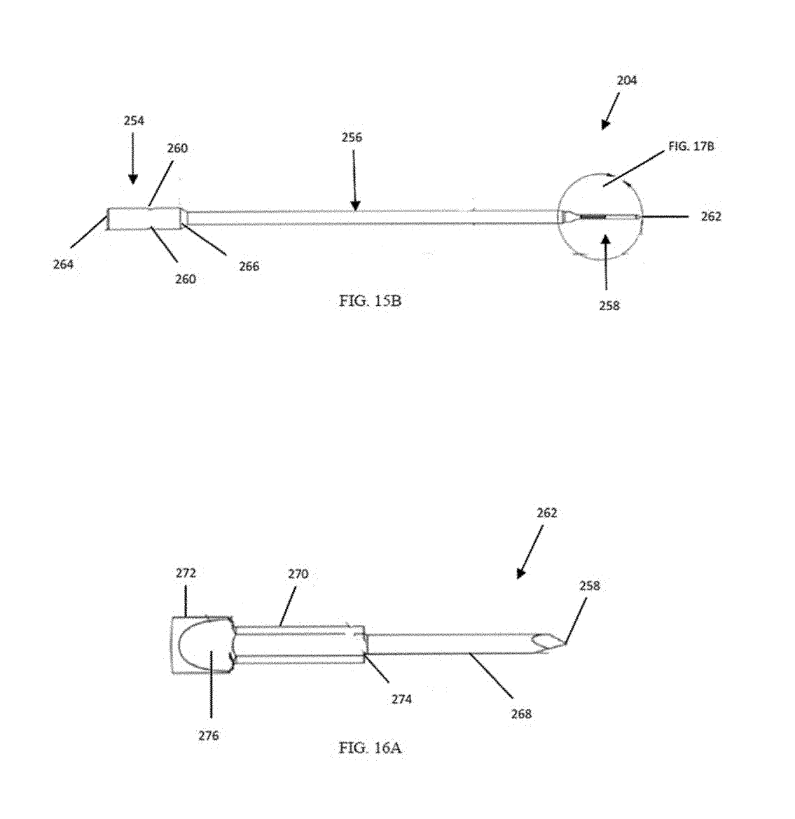

Referring now to FIG. 2C, an end view of the anchor 10 is shown from the proximal surface 16 to the distal surface 20. The thread 24 (FIG. 2B) of the anchor 10 may have an outer surface 44. As was previously described, the anchor 10 may be cannulated such that a bore extends from the proximal surface 16 to the distal surface 20, as may be appreciated in FIG. 2C. As will be described in the FIGS. 3 and 6B, the bore of anchor 10 may include more than one bores 46, 48, and 50. A first bore 46 may be located at the proximal end 18 of the anchor 10. The first bore 46 may be a counter bore. The first bore 46 may allow for clearance of the suture. A second bore 48 may be located adjacent the first bore 46. The second bore 48 may have a non-circular transverse cross-section, such as a square, rectangle, oval, or hexagon. The second bore 48 may allow for insertion of the medial shaft 204 (FIG. 9). A third bore 50 may be located adjacent the second bore 48 and at the distal end 22. The third bore 50 may allow for passage of the medial shaft 204 (FIG. 16A) through the anchor 10 such that a distal tip 262 (FIG. 16A) extends past the distal surface 20. In an exemplary embodiment, the first bore 46 may have a diameter of 2.80 mm and the third bore 50 may have a diameter of 1.32 mm, although other dimensions are contemplated. The second bore 48 may have a width of 0.40 mm and a length of 2.50 mm. The second bore 48 may be radiused at 0.50 mm at the corners of the length and the width.

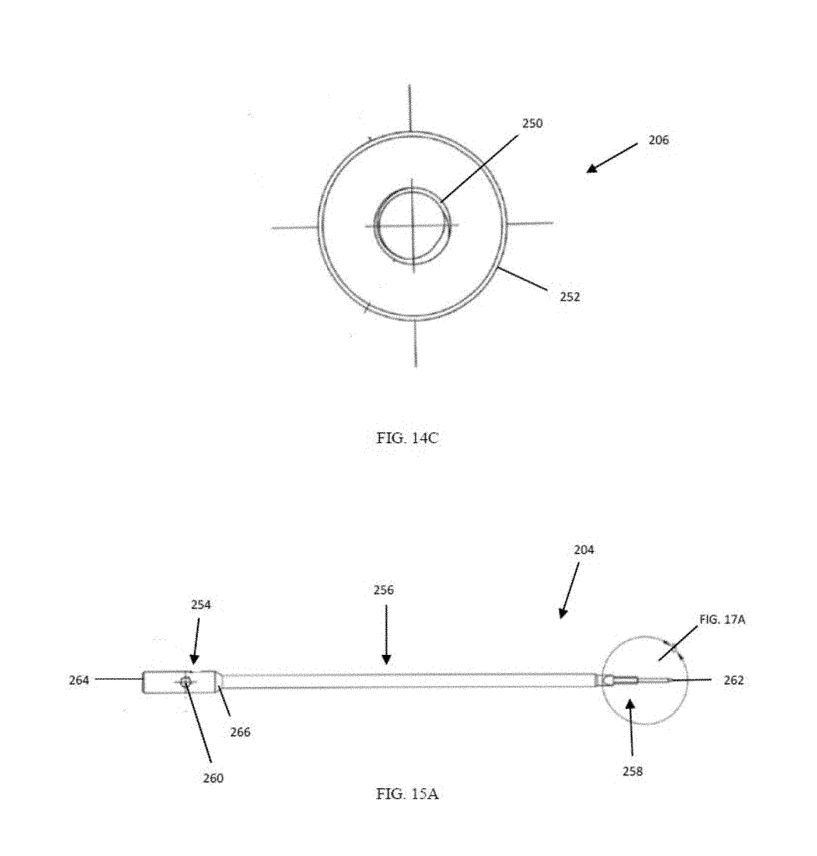

Referring to FIG. 3, a cross-section of anchor 10 with first bore 46, second bore 48, and third bore 50 is shown. The first bore 46, second bore 48, and third bore 50 may be aligned such that they form a continuous bore 52 from the proximal surface 16 to the distal surface 20. The continuous bore 52 may increase in size from the distal surface 20 to the proximal surface 16. The continuous bore 52 may be a multi-dimensional bore due to the multiple dimensions of bores 46, 48, and 50. As previously described the first bore 46 may be a counter bore. The suture opening 28 may be located such that the suture opening 28 intersects first bore 46. The second bore 48 may be adjacent first bore 46. The third bore 50 may be adjacent the second bore 48 and distal surface 20. As may be appreciated from FIGS. 2C and 3, the bore 52 extends from the proximal surface 16 to the distal surface 20 such that anchor 10 is open at both the proximal surface 16 and the distal surface 20. The openness of the anchor 10 may allow for a medial shaft 204 (FIG. 15A) to pass through the anchor 10 from the proximal surface 16 to the distal surface 20. The bore 52 may allow blood to flow through the anchor 10. The threads 24 may begin to taper at a distance 14b from the proximal surface 16. The threads 24 may taper at an angle 14c.

In an exemplary embodiment, the anchor 10 may have a pitch of 2.00 mm or a pitch of 1.50 mm. The distance 14b at which the threads 24 being to taper may be 5.6 mm. The taper angle 14c may be 12 degrees. The length of the first bore 46 may be 2.75 mm. The length of the first bore 46 and the second bore 48 together may be 8.50 mm. The width of each thread 24 may be 0.15 mm near the crest 24a. The angle of each flank 24b may be 120 degrees with respect to the root 24c. The angle of the crest with respect to the root may be 93 degrees. The top of the proximal surface 16 may be chamfered at 30 degrees. The diameter at the root 24c of the anchor 10 may be 4.00 mm or 4.75 mm. The diameter at the crest 24A may be 5.50 mm or 6.00 mm. The diameter at the root 24c may be 4.00 mm when the diameter at the crest 24a is 5.50 mm. The diameter at the root 24c may be 4.75 mm when the diameter at the crest 24a is 6.00 mm. Although, it will be appreciated that these dimensions are exemplary and other dimensions are contemplated.

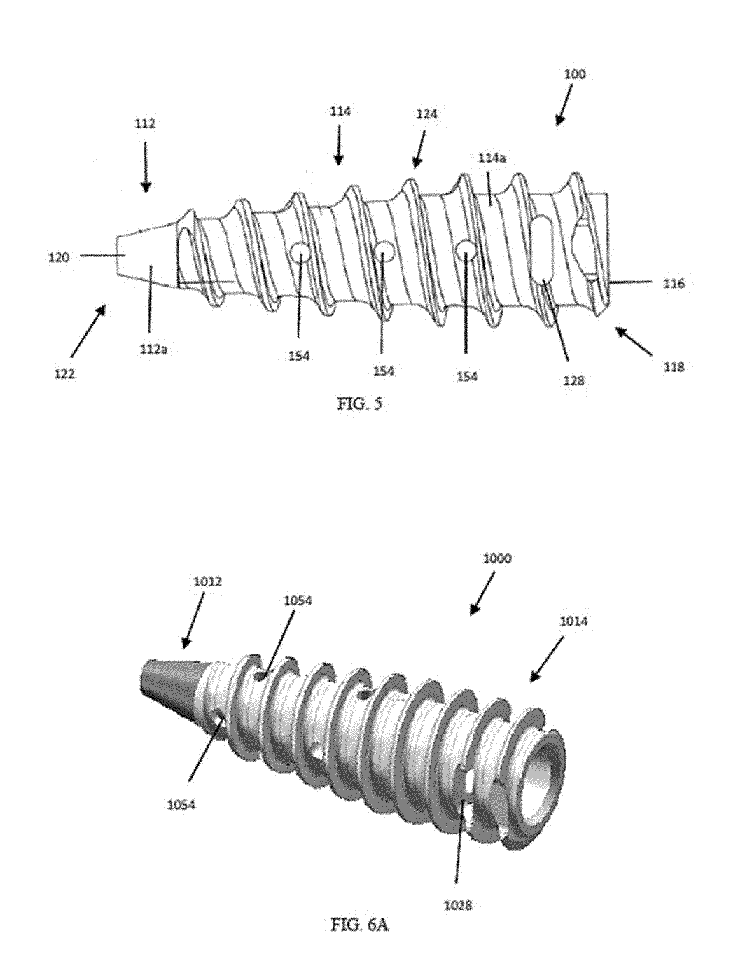

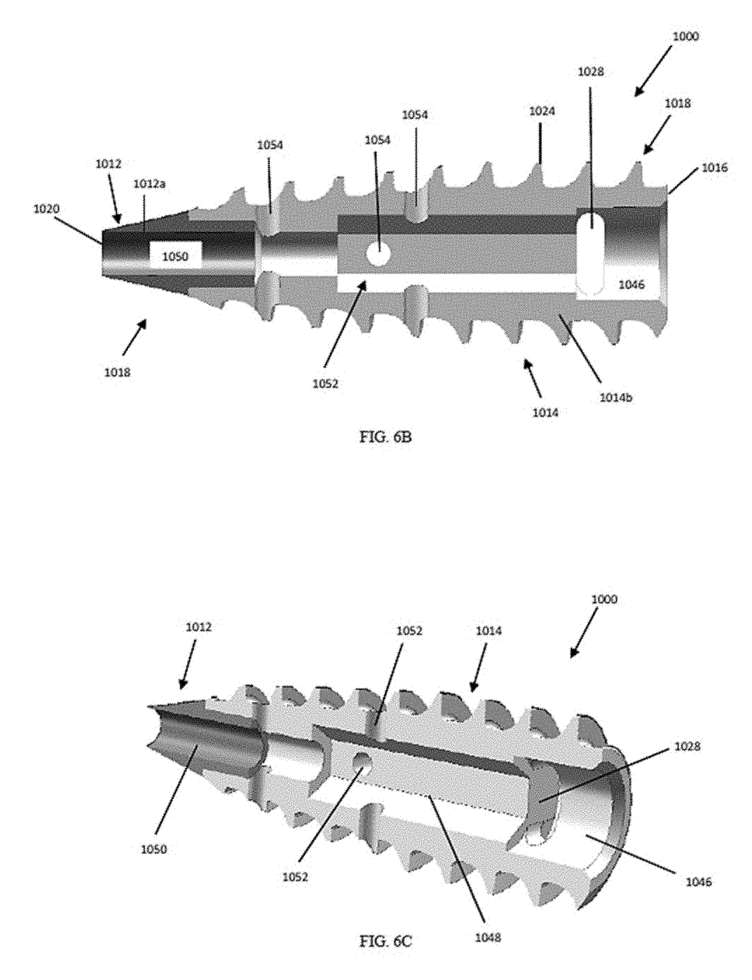

Referring to FIGS. 4-5, an alternate anchor 100 is shown. It may be appreciated that the anchor 100 may be similar or the same as anchor 10 in all respects except for the addition of holes 154 configured to vent blood through the anchor 100. Referring first to FIG. 4, a perspective view of anchor 100 is shown. The anchor 100 may include a threaded portion 114 and a tip portion 112. The anchor 100 may also include holes 154 for venting. The holes 154 may allow blood to travel through anchor 100. Referring now to FIG. 5, and as previously mentioned, the distal surface 20, 120 may be open such that blood is allowed to flow into anchors 10, 100. Holes 154 may provide an additional way for blood to flow through the anchors 10, 100. Although holes 154 are not depicted in anchor 10, it may be appreciated that holes 154 for venting may be provided on anchor 10.

Referring again to FIG. 4, the anchor 100 may include a threaded portion 114 and a tip portion 112. A suture opening 128 may be located in the threaded portion body 114a. Although one suture opening 128 is depicted, it will be appreciated that a second suture opening 128 may be located on a diametrically opposing side of the anchor 100. The suture opening 128 may be large enough to accommodate one or more suture tapes. Thus, the anchor 100 may be able to accommodate two or more suture tapes. The anchor 100 may be able to accommodate a suture tape in each of the two suture openings 128, as will be described herein. Alternatively, more or less suture openings 128 may be provided. The two or more suture openings 128 may be provided at locations other than 180 degrees about the longitudinal axis. That is, for example, the two or more suture openings 128 (or suture openings 28 of anchor 10) may be provided at a 90 degree relationship, or other relative relationship.

Referring to FIG. 5, a side view of the anchor 100 is shown. The anchor 100 may include a threaded portion 114 having threaded portion body 114a and a tip portion 112 having a tip portion body 112a. The anchor 100 may comprise a proximal surface 116 at a proximal end 118 and a distal surface 120 at a distal end 122. The threaded portion 114 may have threads 124. The suture opening 128 and holes 154 may be provided in the threaded portion 114 through the threads 124. The suture opening 128 and/or the holes 154 may be located through a crest, root, or flank of the thread 124. The suture opening 128 may be located at the proximal end 118 of the anchor 100. The holes 154 may be located along a longitudinal length of the anchor 100. Although three holes 154 are depicted, it is understood that more or fewer holes 154 may be provided. Additional holes 154 may be provided on surfaces 90 degrees, 180 degrees, and 270 degrees with respect to the view of FIG. 5. The holes 154 provided on additional surfaces may be provided at the same or different longitudinal and radial locations.

Referring to FIGS. 6A-6D, an alternate anchor 1000 is shown. It may be appreciated that the anchor 1000 may be similar or the same as anchors 10 and 100 in all respects except that the anchor 1000 may include a bore 1048 (FIG. 6C) which is generally hexagonal in transverse cross-section. Referring first to FIG. 6A, a perspective view of anchor 1000 is shown. The anchor 1000 may include a threaded portion 1014 and a tip portion 1012. The anchor 1000 may also include holes 1054 for venting. The holes 1054 may allow blood to travel through anchor 1000. Referring to FIGS. 6B and 6C, and as previously mentioned, the distal surface 1020 may be open such that blood is allowed to flow into anchor 1000. Holes 1054 may provide an additional way for blood to flow through the anchor 1000. Although holes 1054 are not depicted in anchor 10, it may be appreciated that vent holes 1054 may be provided on anchor 10.

Referring again to FIGS. 6B and 6C, the anchor 1000 may include a threaded portion 1014 and a tip portion 1012. A suture opening 1028 may be located in the threaded portion body 1014. Although one suture opening 1028 is depicted, it will be appreciated that a second suture opening 1028 may be located on a diametrically opposing side of the anchor 1000. The suture opening 1028 may be large enough to accommodate one or more suture tapes. Thus, the anchor 1000 may be able to accommodate two or more suture tapes. The anchor 1000 may be able to accommodate a suture tape in each of the two suture openings 1028, as will be described herein. Alternatively, more or less suture openings 1028 may be provided. The two or more suture openings 1028 may be provided at locations other than 180 degrees about the longitudinal axis. That is, for example, the two or more suture openings 1028 (or suture openings 28 of anchor 10 or suture openings 128 of anchor 100) may be provided at a 90 degree relationship, or other relative relationship. As may be appreciated from FIG. 6B, the suture openings 1028 may be staggered along the length of the anchor 1000.

With continued reference to FIGS. 6B and 6C, a cross-sectional side view and cross-sectional perspective view of the anchor 1000 are shown. The anchor 1000 may include a threaded portion 1014 having threaded portion body 1014a and a tip portion 1012 having a tip portion body 1012a. The anchor 1000 may comprise a proximal surface 1016 at a proximal end 1018 and a distal surface 1020 at a distal end 1022. The threaded portion 1014 may have threads 1024. The suture opening 1028 and holes 1054 may be provided in the threaded portion 1014 through the threads 1024. The suture opening 1028 and/or the holes 1054 may be located through a crest, root, or flank of the thread 1024. The suture opening 1028 may be located at the proximal end 1018 of the anchor 1000. The holes 1054 may be located along a longitudinal length of the anchor 1000. More or fewer holes 1054 may be provided than those depicted. Additional holes 1054 may be provided on surfaces 90 degrees, 180 degrees, and 270 degrees with respect to the view of FIGS. 6B and 6C. The holes 1054 provided on additional surfaces may be provided at the same or different longitudinal and radial locations.

Referring again to FIGS. 6B and 6C, a bore 1052 comprising a first bore 1046, second bore 1048, and third bore 1050 is shown. The first bore 1046, second bore 1048, and third bore 1050 may be aligned such that they form a continuous bore 1052 from the proximal surface 1016 to the distal surface 1020. As may be appreciated from FIG. 6C, the first bore 1046 may be a counter bore. The suture opening 1028 may be located such that the suture opening 1028 intersects first bore 1046. The second bore 1048 may be adjacent first bore 1046 and may have a hexagonal shape in lateral cross-section. The hexagonal shape may accommodate a hexagon medial shaft 204. The third bore 1050 may be adjacent the second bore 1048 and distal end 1020. The bore 1052 extends from the proximal surface 1016 to the distal surface 1020 such that anchor 1000 is open at both the proximal surface 1016 and the distal surface 1020. The openness of the anchor 1000 may allow for a medial shaft 204 to pass through the anchor 1000 from the proximal surface 1016 to the distal surface 1020. The bore 1052 may allow blood to flow through the anchor 1000. Referring now to FIG. 6D, an alternate anchor 1000a is shown. Anchor 1000a may be the same in all respects to anchor 1000 except that tip portion 1012a and threaded portion 1014a may be integral such that the anchor 1000a is a single, integral piece.

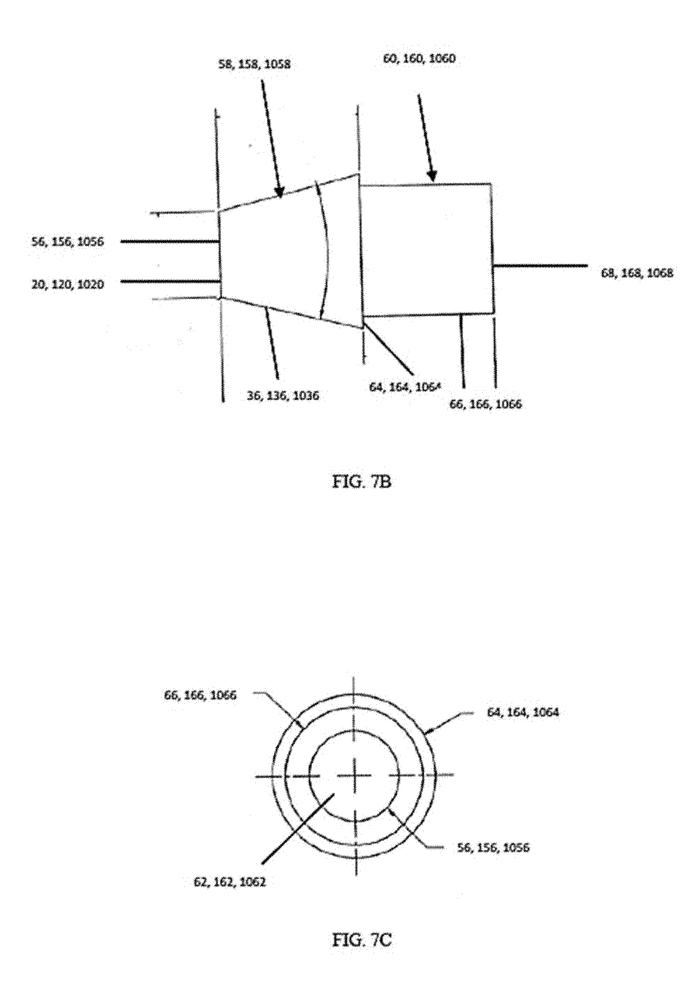

Referring to FIGS. 7A-7C, a tip is shown. This tip may correspond to any of the previously described tip portions 12, 112, 1012 above. For simplicity of description, only tip portion 12 will be described, but it may be appreciated that the tip portion 12, tip portion 112, and tip portion 1012 may be similar or the same. As may be appreciated from FIGS. 7A-7C, the tip portion 12 is depicted as a separate component from the threaded portion 14. When assembled, the second tip portion 60 (FIG. 7A) may be received within a bore 72 (FIG. 8B) of the threaded portion 14. A distal surface 70 (FIG. 8B) of the threaded portion 14 may abut or mate with the tapered portion proximal surface 64 (FIG. 7B). The separate tip 12 may allow for a tip 12 of different material, such as metal, to be installed in an anchor 10 comprising, for example, PEEK, thus customizing the anchor 10 for the specific patient or medical procedure. Alternatively, the tip portion 12 and the threaded portion 14 may be provided as a single, integral, or unitary anchor, such as depicted in, for example, FIGS. 3 and 6D.

With reference first to FIG. 7A, the tip portion 12 may include a first tip portion 58 and a second tip portion 60. The first tip portion 58 may comprise a body 58a and the second tip portion 60 may comprise a body 60a. The first tip portion 58 may be substantially tapered from a tapered portion proximal surface 64 to a tip portion distal surface 56. The first tip portion 58 may comprise the surface 36, as previously described. The second tip portion 60 may be substantially cylindrical, conical, tapered and/or frustoconical, if desired. The second tip portion 60 may extend from a tip portion proximal surface 68 to the tapered portion proximal surface 64. It may be appreciated that a bore 62 extending through tip portion 12 from tip portion proximal surface 68 to tip portion distal surface 56 may align with bore 50, 150, 1050 (FIG. 6B) of the anchors 10, 100, 1000. The alignment of bores may allow for a continuous bore 52, 152, 1052 to be provided in the anchors 10, 100, 1000. Referring to FIG. 7B, it may be appreciated that the tip portion distal surface 56 may align with the anchor distal surface 20. Referring to FIG. 7C, it may be appreciated that the bore 62 extends through the entire tip portion 12. The tapered portion proximal surface 64 may have the largest diameter of the tip portion 12 and the tip portion distal surface 56 may have the smallest diameter of the tip portion 12.

In an exemplary embodiment, the tip portion 12, 112, 1012 may have a length of 4.12 mm. The length of the first tip portion 58, 158, 1058 may be 2.12 mm and the angle of taper may be 27.82 degrees. The diameter of the distal surface 56, 156, 1056 may be 1.30 mm. The length of the second portion 60, 160, 1060 may be 2.0 mm. The diameter of the tapered portion proximal surface 64, 164, 1064 may be 2.35 mm. The diameter of the second portion 60, 160, 1060 may be 1.985 mm. Although it may be appreciated that these dimensions are merely exemplary and other dimensions are contemplated.

Referring to FIGS. 8A-8C, the threaded portion 14, 114, 1014 of anchor 10, 100, 1000 is shown without tip portion 12, 112, 1012 inserted therein. The threaded portion 14, 114, 1014 is substantially the same as previously described with relation to FIGS. 1-6D, without the tip portion 12, 112, 1012. For FIGS. 8A-8C, it may be appreciated how tip portion 12, 112, 1012 is received by threaded portion 14, 114, 1014. For simplicity of description, only threaded portion 14 will be described, but it may be appreciated that the threaded portion 14, threaded portion 114, and threaded portion 1014 may be similar or the same. Although the tip portion 12, 112, 1012 and threaded portion 14, 114, 1014 are depicted and described as two components, it will be understood that the anchors 10, 100, 1000 may be unitary, monolithic, or single piece anchors. As previously described, the threaded portion 14 may comprise a threaded portion body 14a. The threaded portion body 14a may extend from the proximal surface 16 to a threaded portion distal surface 70. The threaded portion 14 may include a bore which extends from the proximal surface 16 to the threaded portion distal surface 70. In an exemplary embodiment, the threaded portion 14, 114, 1014 may have a length of 14.50 mm, although other dimensions are contemplated.

Referring to the cross-section in FIG. 8B taken along the section line B-B of FIG. 8A, showing only a distal portion of the threaded portion 14, 114, 1014 without the tip portion 12, 112, 1012 received therein. Referring also to the cross-section of FIG. 6B showing the threaded 1014 with the tip portion 1012 received therein, it may be appreciated that the bore 50 may align with the bore 62 of tip portion 12. The bore 72 may accommodate the second tip portion 60 of the tip portion 12 through mating of the inner surface of bore 72 and the outer surface of second tip portion 60. As may be appreciated, during assembly, the tip portion 12 may be installed into the threaded portion 14 such that the threaded portion distal surface 70 may mate with the tapered portion proximal surface 64 to assemble the anchor 10. The tip portion 12 may be assembled during manufacturing with the threaded portion 14 such that it is supplied to the user as a single anchor 10. Alternatively, the tip portion 12 may be assembled into the threaded portion 14, by the user prior to the medical procedure. In either case, the tip portion 12 may be press fit into the threaded portion 14. Alternatively, the tip portion 12 may be retained in the threaded portion 14 with a friction fit, adhesive, taper fit and/or threaded engagement, if desired. Referring to the end view of FIG. 8C, the outer surface 44 and bores 46, 48, and 50 may be seen. In an exemplary embodiment, the length of the bore 72 may be 2.00 mm and the diameter of bore 72 may be 1.985 mm, although other dimensions are contemplated.

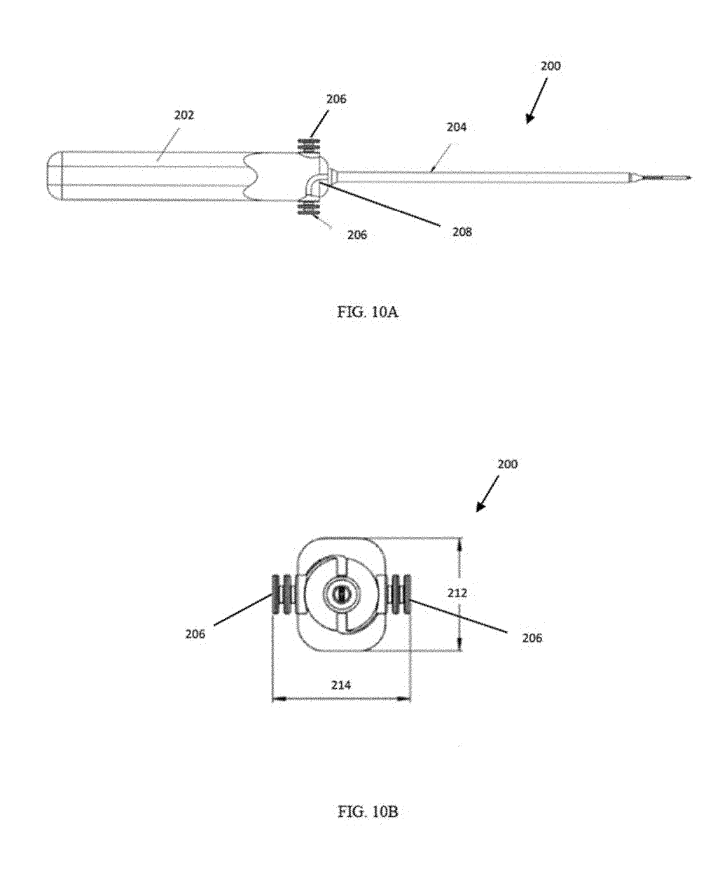

Referring to FIGS. 9-11, an awl 200 for insertion through the anchor 10, anchor 100, or the anchor 1000 is shown. Referring to FIG. 9, a perspective view of the awl 200 is shown. The awl 200 is preferably configured to mate or engage with an interior surface of the anchor 10, 100, 1000. The mating engagement allows for the awl to operate as a drive mechanism, transmitting torque applied to the handle to the anchor. The awl 200 may include a handle 202 and a medial shaft 204. The medial shaft 204 may be attached to the handle 202 with one or more handle pins 206, as will be described herein. Referring now to FIG. 10A, a top view of the awl 200 is shown. The handle 202 of the awl 200 may include channels 208. The channels 208 may hold a suture tape (not shown) when the awl 200 is inserted into the anchor 10, 100, 1000 for insertion into a patient, as will be described herein. In an exemplary embodiment, the awl 200 may have a length of 309.75 mm, although other dimensions are contemplated. Any or all of the components of the awl, such as medial shaft 204 and/or the handle 202 may comprise a metal.

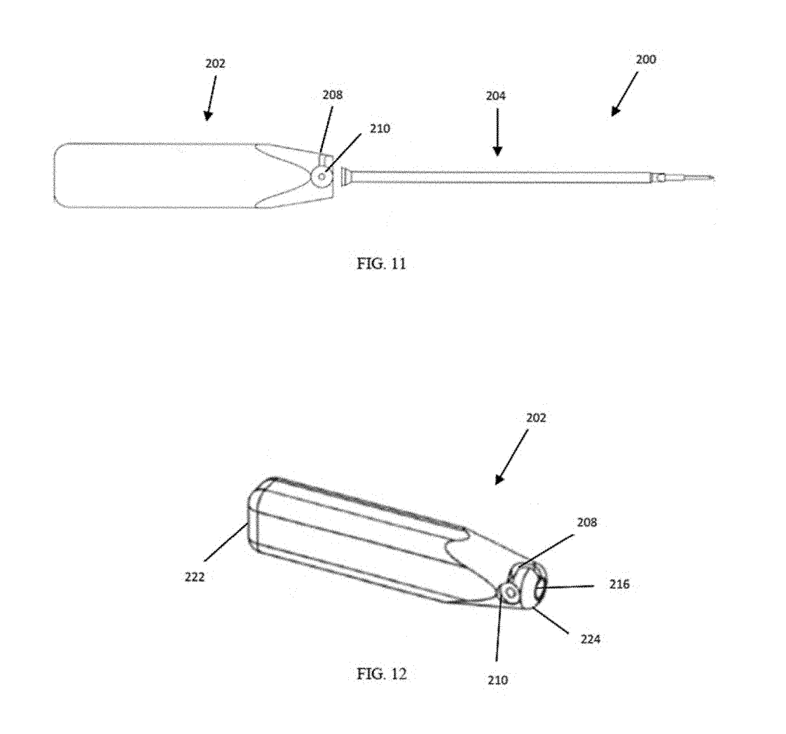

With reference to FIG. 10B, an end view of the awl 200 is shown. It may be appreciated from this view that the handle 202 may have a generally rectangular shape. The handle 202 may have first dimension 212. The handle 202 may further have an awl width 214 which extends between the handle pins 206. With reference to FIG. 11, an exploded side view of the awl 200 is shown. The handle 202 of the awl 200 may include a countersunk bore 210. The countersunk bore 210 may receive the handle pins 206 (FIG. 10A). In an exemplary embodiment, the first dimension 212 may be 30 mm and the awl width 214 may be 36 mm, although other dimensions are contemplated.

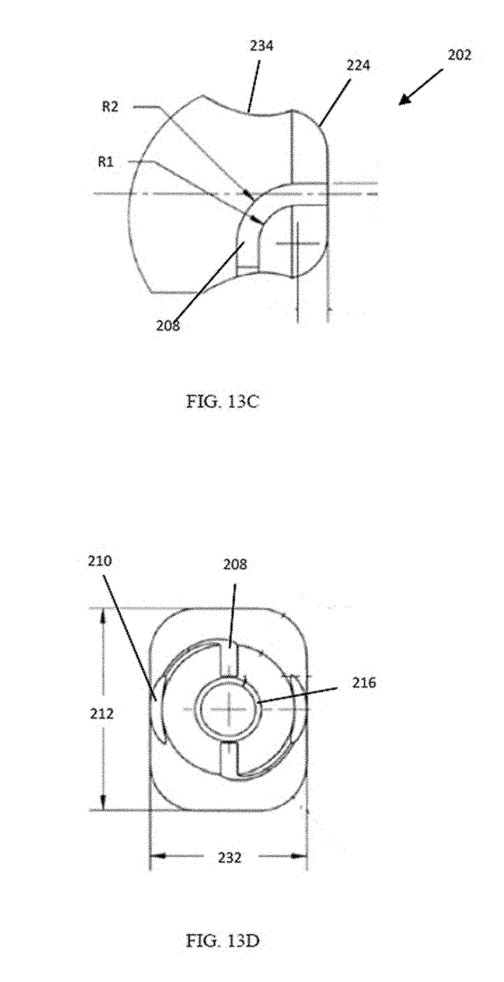

FIGS. 11 and 13A-13D show views of the handle 202 of the awl 200. The handle 202 may comprise a bore 216 at the distal surface 224 for receiving the medial shaft 204 (FIG. 10A). The channel 208 is shown to extend from an end of the handle 202 radially around the outer surface of the handle 202 to the countersunk hole 210. Thus, a suture tape may extend from the anchor 10, 100, 1000 along the medial shaft 204 (FIG. 10A) and into the channel 208. The suture tape may then be wound around the handle pins 206 and extend upward to a proximal surface 222 of the handle 202. The suture tape, extending through the suture opening and around the handle pin 206 may secure the anchor 10 (or anchors 100, 1000) onto the awl 200. Alleys may be provided in the handle 202 for extending the suture tapes within past the handle pins 206 to the proximal end of the handle 202.

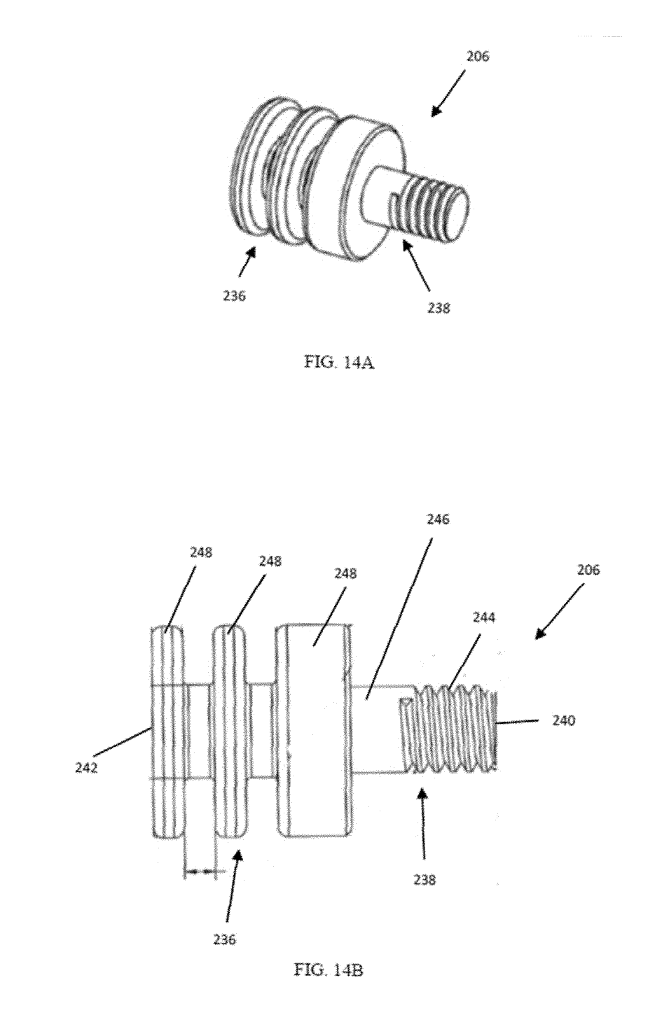

FIGS. 14A-14C show views of the handle pin 206. As was previously described, one or more handle pins 206 may be provided with the handle 202 for securing the medial shaft 204 within a bore 216 (FIG. 11) of the handle 202. Referring first to FIG. 13A, the handle 202 may include a tapered portion 218 having a tapered surface 226. The handle 202 may have a non-tapered portion 220. Although the non-tapered portion 220 is depicted and described as rectangular, it may be appreciated that any shape, such as square, oval, circle, etc., may be provided. The handle 202 may taper such that the distal surface 224 has a first dimension 228 smaller than a dimension 2R of the proximal surface 222.

With reference to FIG. 13B, the handle 202 is shown rotated 90 degrees from the view of FIG. 13A. The handle 202 may have a dimension 232 which is less than the dimension 2R (FIG. 13A). Thus, the handle 202 may be generally rectangular, as may be appreciated from FIG. 13D. Although, the dimension 212 may be the same as the dimension 232 or the dimension 212 may be less than the dimension 232. The handle 202 may have a length 230. The handle 202 may further have a radiused portion 234.

Referring now to FIG. 13C, a detailed view of the proximal end of the handle 202 is shown. The channel 208 may be curved such that the channel has an inner radius R1 smaller than an outer radius R2. The curved channel 208 may allow for a suture to extend within, and in various embodiments the channel may be open to the exterior to allow for “top-loading” of the suture into the channel and/or may include inwardly tapered or curved walls to asset with retention of the suture therein. Although only one channel 208 is depicted, it may be appreciated that a second channel 208 may be provided on a diametrically opposing surface of the handle 202. In this manner, the handle 202 may accommodate a second suture tape. Although two channels 208 are shown and described, more or fewer channels may be provided. The shape of channel 208 may be any shape able to accommodate a suture tape.

In an exemplary embodiment, the handle 202 may have a length of 135 mm. The tapered portion 226 may have a taper angle of 20 degrees. The countersunk bore 210 may have a countersunk diameter of 10.30 mm and a through hole diameter of 4.22 mm. The countersunk bore 210 may be located a distance 9.25 mm from the distal surface 224. The distal surface 224 may have a diameter of 18 mm. The inner radius R1 of channel 208 may be 4.50 mm and the outer radius R2 may be 7.00 mm. The dimension 232 may be 23 mm. The handle 202 may be radiused between the dimensions 212 and 232 at 7 mm. The diameter of bore 216 may be 7.94 mm. Although, it may be appreciated that other dimensions are contemplated.

FIGS. 14A-14C show views of the handle pin 206. The handle pin 206 may have a handle portion 236 and a stem portion 238. Referring to FIG. 14B, the handle pin 206 may extend from a first surface 240 to a second surface 242. The stem portion 238 may include a threaded portion 244 and a non-threaded portion 246. The threaded portion 244 may be received in a corresponding threaded portion of countersunk hole 210 (FIG. 13A). The handle portion 236 may include enlarged portions 248. The enlarged portions 248 may facilitate grasping. The enlarged portions 248 may allow for a suture tape to be wrapped around. Referring to FIG. 14C, an end view of the handle pin 206 is shown. The handle pin 206 may have an outer surface 250 of the stem portion 238 which is smaller than an outer surface 252 of the handle portion 236.

In an exemplary embodiment, the length of handle pin 206 may be 16.50 mm. The diameter of the stem portion 238 and the handle portion 236 may be 4.50 mm. The diameter of the enlarged portions 248 may be 10.25 mm. The distance between the enlarged portions 248 may be 1.5 mm. The width of the first and second enlarged portions 248 may be 1.50 mm. The width of the third enlarged portion 248, adjacent the step portion 238, may be 3.50 mm. The length of the stem portion 238 may be 7.00 mm. The length of the threaded portion 244 may be 4.00 mm. Although, it may be appreciated that other dimensions are contemplated.

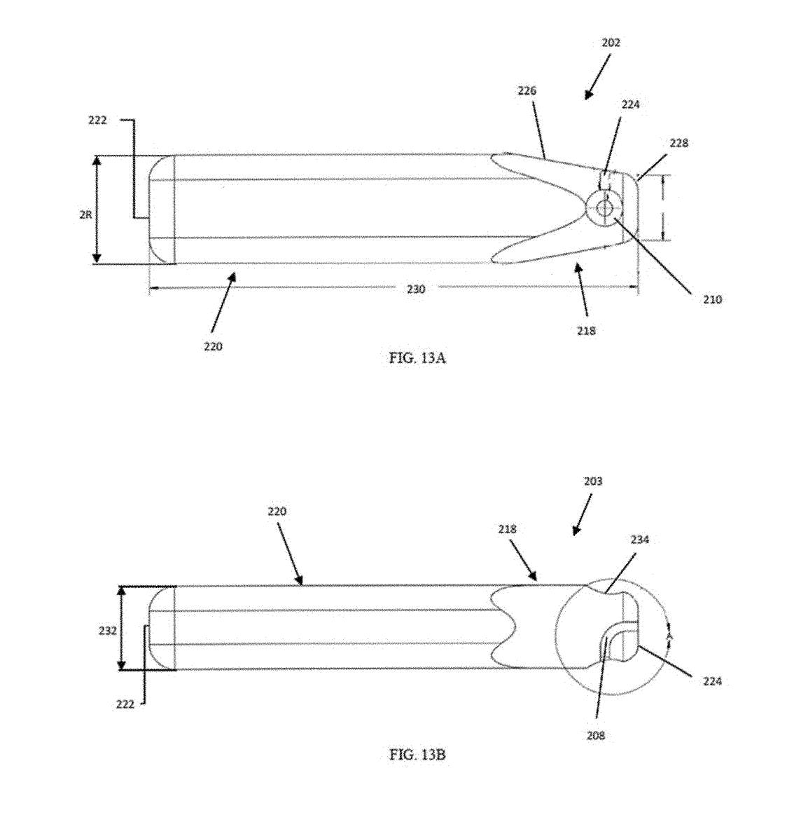

FIGS. 15-17 show views of the medial shaft 204 of the awl 200. The medial shaft 204 may be sized and shaped to be received within the central bore of the anchor 10 or the anchor 100 such that a distal tip 262 (FIG. 15A) extends past the distal surface 20 (FIG. 2A). Referring first to FIG. 15A, the medial shaft 204 may have a first portion 254, a second portion 256, and a third portion 258. The first portion 254 may be received within the bore 216 (FIG. 12) of handle 202.

With continued reference to FIG. 15A, the first portion 254 may include holes 260 which may be aligned with countersunk holes 210 of the handle 202. The aligned holes 260 and 210 may receive the handle pin 206 for securing the medial shaft 204 to the handle 202. The medial shaft 204 may extend from the distal tip 262 to a proximal surface 264. The medial shaft 204 may further have a taper 266 from the first portion 254 to the second portion 256. In an exemplary embodiment, the holes 260 may have a diameter of 3.45 mm and may be located a distance of 16 mm from the proximal surface 264, although other dimensions are contemplated.

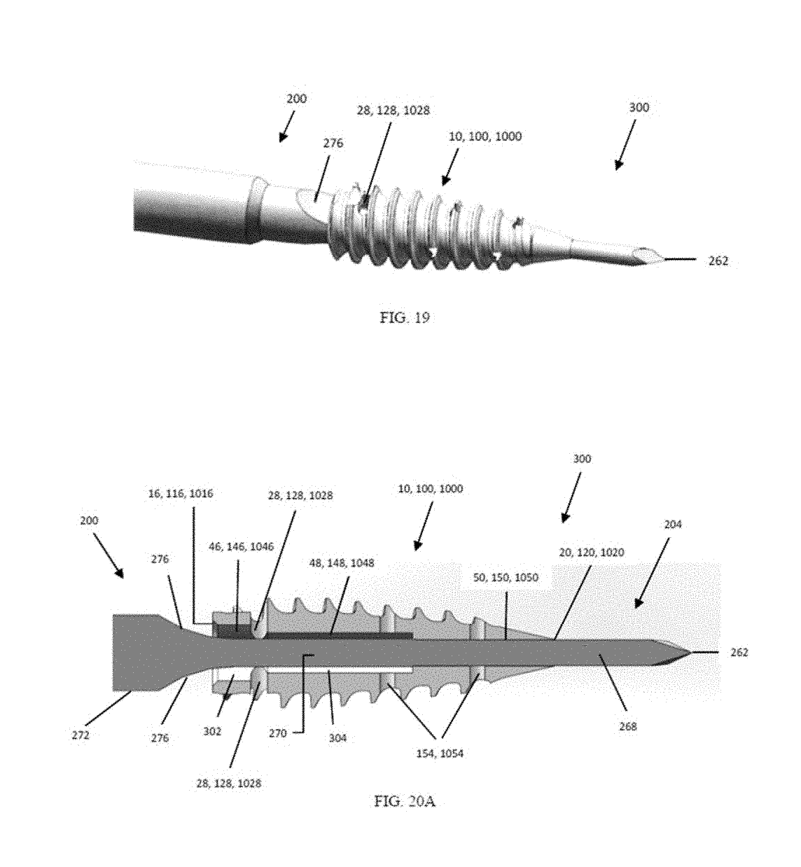

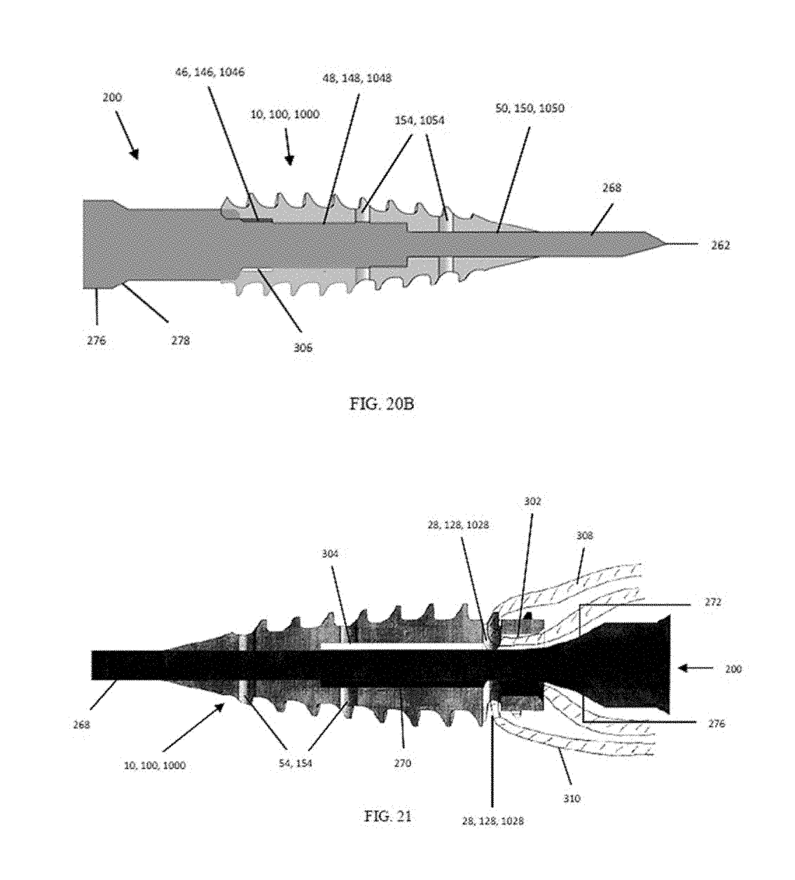

A detailed view of the third portion 258 of FIG. 15A is shown in FIG. 16A. With reference to FIG. 16A, the third portion 258 may comprise three sections, a first section 268, a second section 270, and a third section 272. The first section 268 may have a generally circular transverse cross-section that may be received with the bore 50 (FIG. 3) of anchor 10 or anchor 100. The first section 268 may extend from the distal tip 262 to the second section 270. The medial shaft 204 may have a gradual curvature 274 between the first section 268 and the second section 270. The second section 270 may have a generally non-circular transverse cross-section shaped to match the non-circular transverse cross-section of bore 48 (FIG. 3). Thus, the second section 270 of medial shaft 204 may be received in the bore 48 to provide a secure fit of the medial shaft 204 within the anchor 10 or anchor 100. The non-circular transverse cross-section of second section 270 may be an oval, rectangle, or hexagon, for example. The transition between the second section 270 and the third section 272 may include a cutout 276. The cutout 276 may such that, when inserted into an anchor 10 or anchor 100, there is a space between the medial shaft 204 and the wall of bore 46 (FIG. 3). This space may allow for a suture tape to pass through the suture opening 28 (FIG. 3) and past the medial shaft 204, as will be explained with reference to FIG. 19. In an exemplary embodiment, the distal tip 262 may be pointed at an angle of 15 degrees on all sides, although other embodiments are contemplated.

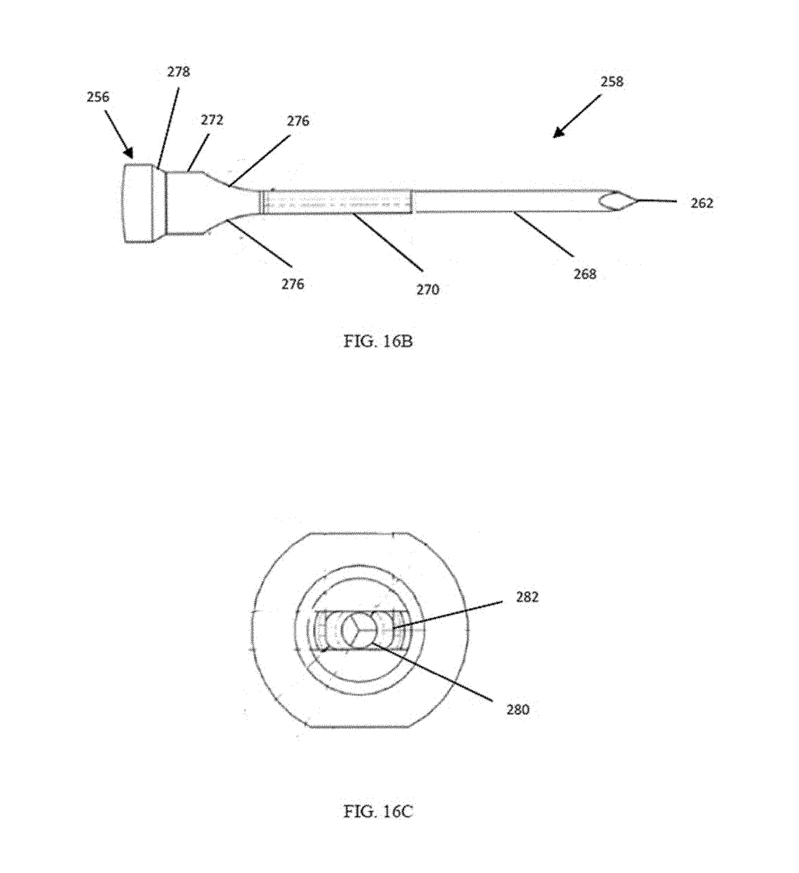

With reference now to FIG. 15B, the medial shaft 204 of FIG. 15A is shown rotated 90 degrees. It may be appreciated from FIG. 15B, that the holes 260 are located on two opposing surfaces of first portion 254. A detailed view of the third portion 258 of FIG. 15B is shown in FIG. 16B. That is, the detailed view of FIG. 16B is the third portion 258 of FIG. 16A, rotated 90 degrees. The second portion 256 of medial shaft 204 may have a tapered section 278 extending to the third portion 258, specifically to the third section 272 of the third portion 258. There may be two cutouts 276 located on opposing surfaces of the third section 272. The cutouts 276 may be located such that the cutouts 276 extend from the generally circular transverse cross-section of section 272 to the long side 280 (FIG. 16C) of second section 270 (FIG. 17). That is, referring to FIGS. 16C and 17, where section 270 is ovular or rectangular, it may comprise a long side 280 and a short side 282. The long side 280 and the short side 282 may be configured such that they engage with the bore 48 to provide driving engagement between the awl 200 and the anchor 10, 100. The cutouts 276 may transition from the third section 272 to the long side 280 of the second section 270. FIG. 17 is a perspective view of a part of the medial shaft 204. The non-circular transverse cross-section of second section 270 may be appreciated in this view. In an exemplary embodiment, the first portion 254 may have a length of 27 mm. The second portion 256 may have a diameter of 4.76 mm. The overall length of the medial shaft 204 from the proximal surface 264 to the distal tip 262 may be 200 mm. The third section 272 may have a diameter of 3.80 mm. The first section 268 may have a diameter of 1.3 mm and a length of 14 mm. The first section 268 and the second section 270 together may have a length of 23 mm. The short side 282 may have a dimension of 1.40 mm and the long side 280 may have a dimension of 2.50 mm. Although, it may be appreciated that other dimensions are contemplated.

It may be appreciated from the foregoing that the awl 200 may be inserted into the anchor 10, 100 for driving the anchor 10, 100 into bone. As will be described in more detail to follow, the awl 200 may be inserted into bore 52, 152 of the anchor 10, 100. Upon insertion, the third portion 258 will extend through the third bore 50 with the distal tip 262 extending past the distal surface 20 of the awl. The second section 270 of the third portion 258, comprising the rectangular or ovular shape, may engage be located in the second bore 48, 148 of the anchor 10, 100. Once inserted, the complementary shapes of the second section 270 and second bore 48, 148 may allow for the awl 200 to be securely engaged with the anchor 10, 100. Thus, during use, the handle 202 of the awl 200 may be turned and the rotation may be translated to the anchor 10, 100 due to the driving engagement between the second section 270 and the second bore 48, 148. In this manner, once the tip 262 is punched into the bone to create a pilot hole, the anchor 10, 100 may be advanced into the bone through rotation of the handle 202 which translates to rotation of the anchor 10, 100.

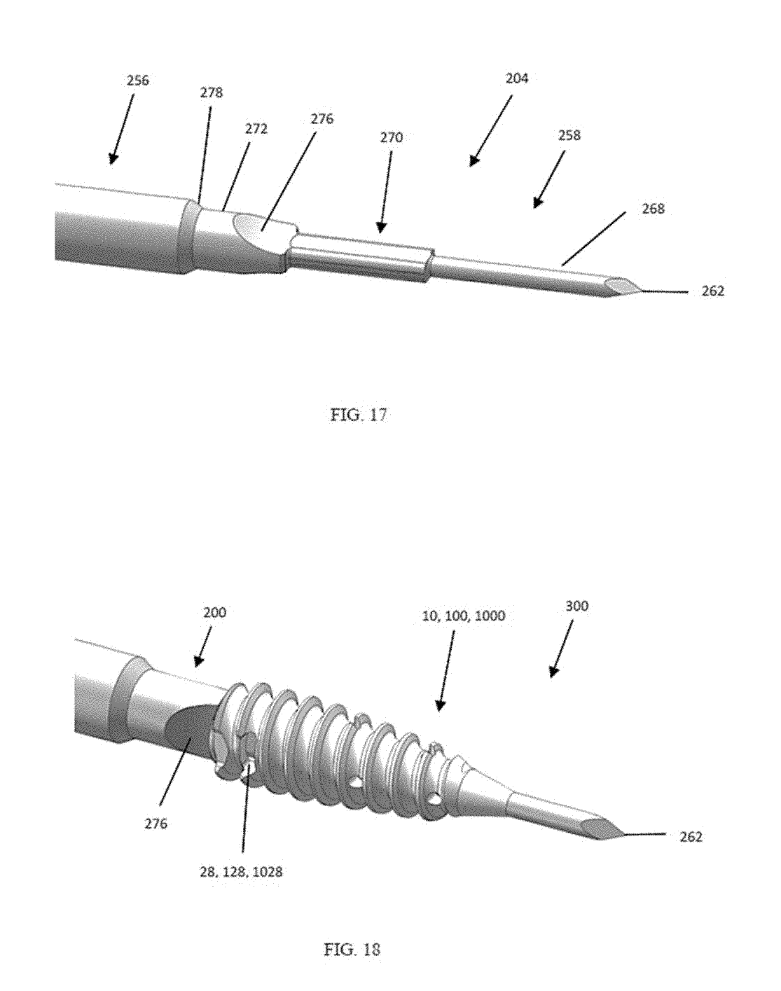

Although FIGS. 9-17 depict and describe an awl 200 having a second section 270 that is elliptical, ovular, or rectangular for use with anchor 10 or anchor 100. It may be appreciated that where anchor 1000 is selected, the second section 270 may have a transverse cross-section that is generally hexagonal to mate with bore 1048 of anchor 1000. All other aspects of the awl 200 may be the same. Where a hexagonal anchor 1000 and awl 200 are provided, gap 304 may be omitted as a tight fit may be provided between the outer surface of awl 200 and inner surface of anchor 1000. It may also be appreciated that the mating surfaces of the anchor and awl, whether hexagonal, rectangular, elliptical, or otherwise, allow for a drive mechanism in the tool 300 (FIG. 18). That is, the mating surfaces allow for torque provided to the handle 202 to be translated to the anchor 10, 100, 1000 for advancing into the bone. Therefore, as described with relation to anchors 10, 100, the complementary shapes of second section 270 and the bore 1048 may allow for the awl 200 to be securely engaged with the anchor 1000. Thus, during use, the handle 202 of the awl may be turned and the rotation may be translated to the anchor 1000 due to the driving engagement between the second section 270 and the second bore 1048. The anchor 1000 can thus be advanced into the bone in the same or similar manner as with respect to anchor 10, 100.

FIG. 18 shows a perspective view of a tool 300 which includes the awl 200 inserted through any of anchors 10, 100, 1000. The tool awl 200 and anchor 10, 100, 1000 are configured to cooperatively engage each other in accordance with the principles of the invention. The tool 300 may provide a one-step tool which may advance an anchor 10, 100, 1000 into bone after a pilot hole has been punched by distal tip 262, in a single insertion step. The tool 300 may also provide the ability to carry two suture tapes in the two suture openings 28, 128, 1028. As shown, the tool 300 may include the awl 200 and one of the anchor 10, the anchor 100, or the anchor 1000. FIG. 18 shows a partial perspective view of the tool 300. As may be appreciated from these views the cutouts 276 are aligned with the side of anchor 10, 100, 1000 with the suture opening 28, 128, 1028. The distal tip 262 of the awl 200 may extend past the distal surface 20 of the anchor 10, 100, 1000. The cutouts 276 may be aligned with the suture openings 28, 128, 1028 to allow for the suture tape to extend through the suture openings 28, 128, 1028 and along the cutout 276 to a proximal end of the awl 200.

FIG. 19 shows a partial cross-sectional view of the tool 300. Referring to FIG. 19, the tool 300 is shown with the awl 200 inserted into an anchor 10, 100, 1000 before a suture tape has been installed in the anchor. When installed, a gap or annular space 302 may be created in bore 46, 146, 1046 between the medial shaft 204 and the anchor 10, 100, 1000. The medial shaft 204 may have a smaller outer dimension than the dimension of bore 46, 146, 1046.

Gap 302 is created due to the difference in dimensions of medial shaft 204 and bore 46, 146, 1046. The gap 302 may allow for a suture, preferably, a suture tape to be inserted into suture opening 28, through gap 302 and along cutout 276. This may allow for the suture tape to extend from the anchor 10, 100, 1000 along the length of the awl 200. Another gap 304 may be created due to a difference in dimension of medial shaft 204 and bore 48, 148, 1048. The gap 304 may allow for blood to flow through holes 154 and up through the inner bore 52 of the anchor 10, 100, 1000. With reference to FIG. 19, it may be appreciated that the medial shaft 204 has a tight fit within the bore 52 of the anchor 10, 100, 1000 such that the awl 200 is secured within the anchor 10, 100, 1000 when in use for a medical procedure. A gap 306 may be present in bore 46 to allow for blood to flow through the anchor 10, 100, 1000.

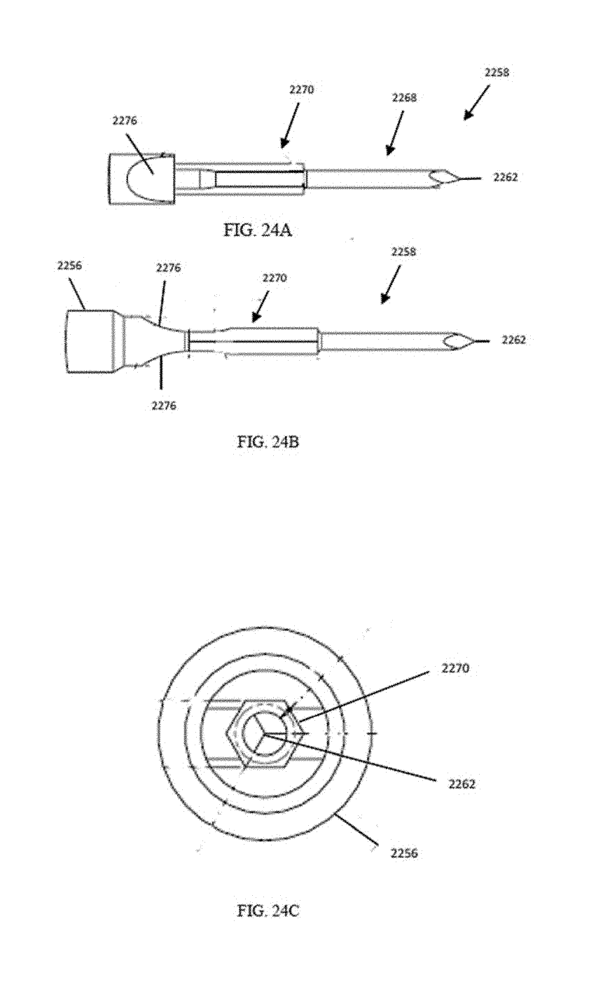

FIG. shows a partial cross-sectional view of the tool 300 with suture tapes 308 and 310 installed in the suture openings 28, 128, 1028. As may be appreciated, the suture tape 308 may extend from an exterior of the anchor 10, 100, 1000 through the suture opening 28 and into gap 302. The suture tape 308 may then extend along the cutout 276. The suture tape may extend from the cutout 276 along the medial shat 204 to the channels 208 (FIG. 12). Suture tape 310 may be extend through anchor 10, 100, 1000 in a similar or the same manner as described with relation to suture tape 308. Thus, it may be appreciated that two suture tapes 308 and 310 may be installed in the tool 300. Although two suture tapes 308 and 310 are depicted installed into two suture openings 28, it may be appreciated that more or fewer suture tapes and/or more or fewer suture openings may be provided. FIG. 22 shows a perspective view of the anchor 10, 100, 1000 with suture tapes 308 and 310 installed, but with the awl 200 removed for clarity. As may be appreciated, the suture tapes 308 and 310 extend from an outer surface of the anchor through the bore 52 and out an opening on the proximal surface 16 of the anchor.

FIG. 22 shows a perspective view of suture tapes 308, 310 installed along the anchor 10, 100, 1000 and awl 200. As may be appreciated, the suture tapes 308, 310 extend along the medial shaft 204 and along the handle 202. Alleys 312 may be provided on opposing surfaces of the handle 202 to accommodate the suture tapes 308, 310. The suture tapes 308, 310 may extend through alleys 312. The suture tapes 308, 310 may be wrapped around an end of the handle 202 to secure the anchor 10, 100, 1000 to the awl 200. When secured, the suture tapes 308, 310 may assist in retaining anchor 10, 100, 1000 onto the medial shaft 204. Thus, when the suture tapes 308, 310 are removed from the awl 200 to be secured to tissue or tendon in the patient, the awl 200 may be unsecured from the anchor 10, 100, 1000. When unsecured from the anchor 10, 100, 1000, the awl 200 may be pulled by handle 202 to remove the awl 200 from the anchor 10, 100, 1000. In this manner, it may be appreciated that the medial shaft 204 and handle 202 may be reused with another anchor. As may be appreciated from the foregoing disclosure, the awl 200 may be reusable or non-disposable. That is, a user may install the awl 200 in a desired anchor 10, 100, 1000 and install the anchor into bone during a medical procedure. The awl 200 may then be removed from the anchor 10, 100, 1000. Once removed, the awl 200 may be used with a second anchor 10, 100, 1000. One or both of the handle 202 and medial shaft 204 may be non-disposable or reusable. Although the foregoing description describes suture tapes 308, 310, it may be appreciated that any suture may be used, such as, for example, a suture thread.

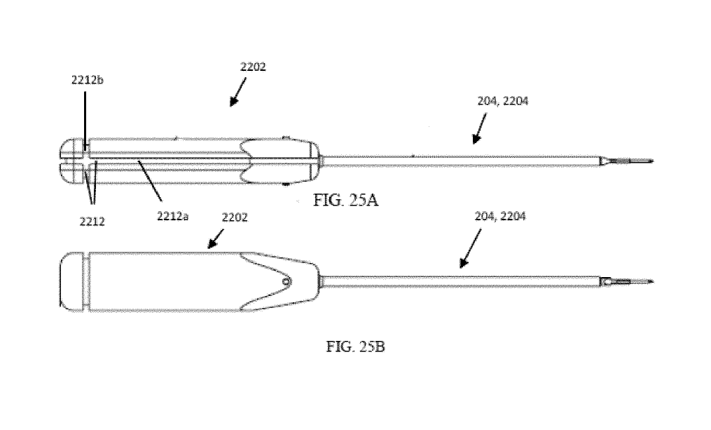

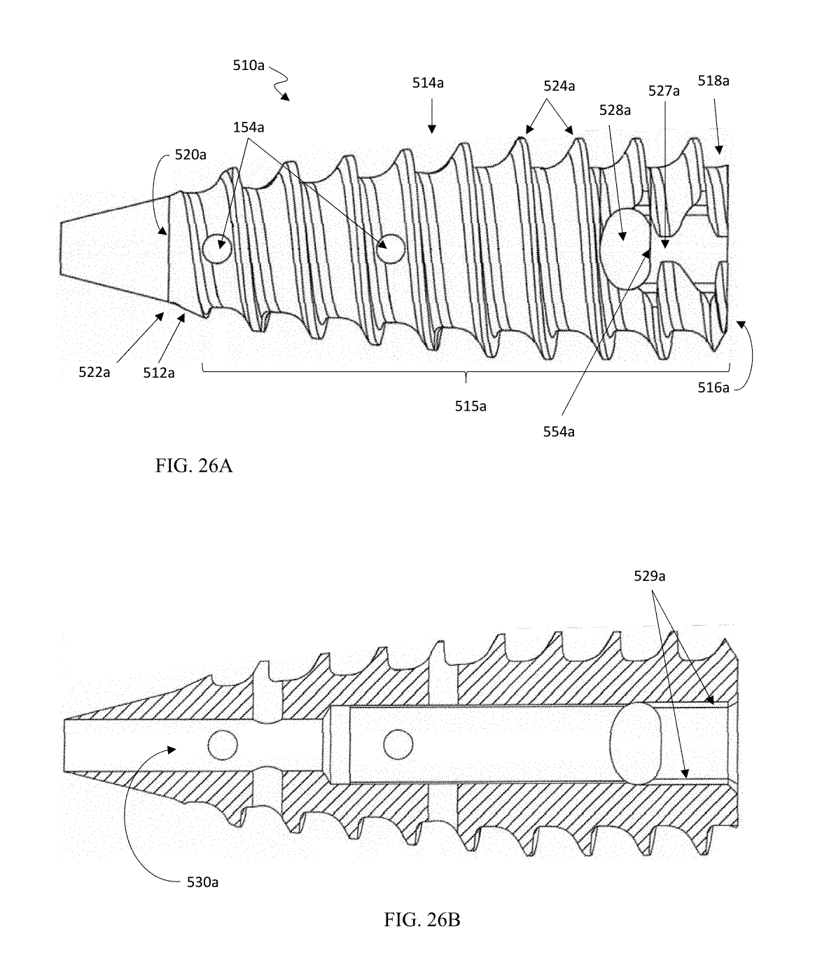

Referring to FIGS. 23-24, views of an alternate medial shaft 2204 are shown. The medial shaft 2204 may be the same in all respects to the medial shaft 204 except for the second section 2270 of the third portion 2258 which may differ in shaft from the second section 270 of the third portion 258 of the medial shaft 204 (FIGS. 16A-16B). As shown in FIGS. 24A and 24B, the medial shaft 2204 may include a second portion 2256 and a third portion 2258. The third portion 2258 may include a first section 2268 including a distal tip 2262. The third portion 2258 may further include cutouts 2276 (FIGS. 24A and 24B). The cutouts 2276 may allow for a suture to extend into a space between the medial shaft 2204 and an anchor 10, 100, 1000, similar to cutouts 276. The medial shaft 2204 may differ from the medial shaft 204 at the second section 2270. The second section 2270 may include a generally hexagonal shape in transverse cross-section (FIG. 24C). The hexagonal outer surface of second section 2270 may mate with a corresponding hexagonal bore on anchor 10, 100, or 1000.

In an exemplary embodiment, the medial shaft 2204 may have a length of 200 mm. In an exemplary embodiment, a length from the distal tip 2262 to the beginning of the cutout 2276 may be 22.15 mm. In an exemplary embodiment, a length from the distal tip 2262 to the beginning of the second section 2270 may be 12.15 mm. In an exemplary embodiment, a radius of the cutout 2276 may be 6.35 mm. Although other dimensions of medial shaft 2204 are contemplated. Any or all of the components of the awl, such as medial shaft 2204 and/or the handle 2202 may comprise a metal.

As may be appreciated from FIGS. 16A-16B and FIGS. 23A-23B, the medial shafts 204 and 2204 have holes (260 in FIG. 16A) for securing to handle 202. However, it may be appreciated that the holes may be omitted and other connection means may be provided. Such connection means may be an AO connect or Hudson connect, as are known in the art. Where holes 260 are omitted, pins 206 may also be omitted and the suture tapes may be secured around channels within the periphery of the handle. Alternatively, the handle (202 in FIG. 9) may be omitted and a connection provided on the medial shaft 204, 2204 for connection to a power tool, such as a drill. Where an AO connect or Hudson connect are provided, one may appreciate the medial shaft 204, 2204 may attach directly to the handle 202, 2202 or to the power tool, via the AO connect or Hudson connect. Other connections between the handle or power and the medial shaft are contemplated.

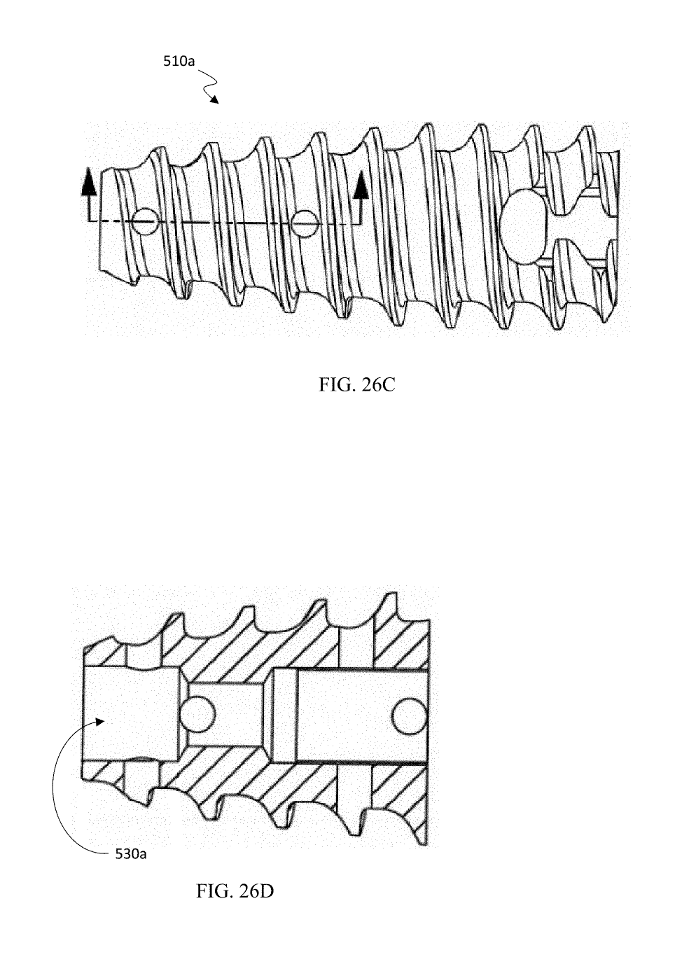

An exemplary embodiment of a handle with pins 206 omitted is depicted in FIGS. 25A and 25B. FIG. 25A shows a handle 2202 attached to medial shaft 2204, however the handle 2202 may also be attached to medial shaft 204. FIG. 25B shows the handle 2202 with alleys 2212a able to accommodate suture tapes, wherein the suture tapes may extend up longitudinal alleys 2212a and be wrapped around the periphery of the handle 2202 in radial alleys 2212b. When secured, the suture tapes may assist in retaining anchor 10, 100, 1000 onto the medial shaft 204, 2204. Thus, when the suture tapes are removed from the handle 202, 2202 to be secured to tissue or tendon in the patient, the handle 202, 2202 and the medial shaft 204, 2204 may be unsecured from the anchor 10, 100, 1000. When unsecured from the anchor 10, 100, 1000, the medial shaft 204, 2204 may be pulled by handle 202, 2202 to remove the same from the anchor 10, 100, 1000. In this manner, it may be appreciated that the medial shaft 204, 2204 and handle 202, 2202 may be reused with another anchor.

With the tool 300 described, the use of the tool 300 may now be appreciated. The tool 300 may be used for a medical procedure, such as rotator cuff repair. The anchor 10, 100, 1000 of tool 300 may be self-punching and allow for easy insertion into bone and tissue of a patient during the medical procedure. As may be appreciated from the foregoing disclosure, a sharp tip of the awl may extend past a distal end of the anchor. The sharp tip of the awl may be metal. The metal, sharp tip of the awl may facilitate forming of a hole in the bone. The awl, with anchors and suture tapes installed, may be impacted into the bone such that the sharp tip creates a pilot hole in the bone. The handle of the awl may then be rotated such that the anchor is advanced into the pilot hole created by the tip of the awl. The tapered, non-threaded shape of the tip portion 112 of the anchor may be impacted into the pilot hole. The tapered, non-threaded portion of the anchor allows for the anchor to be guided into the pilot hole. As the handle of the awl is rotated, the threaded outer surface of the anchor allows for the anchor to be advanced into the bone. When the anchor is fully inserted into the bone, the awl may be removed. Since the awl is removed after insertion of the anchor, the awl may be cleaned and sanitized and reused in a subsequent medical procedure. With the awl removed, the suture tapes can be secured to the tissue or tendons. The cannulation of the anchor and the vent holes allow for blood and biologic healing products to enter the anchor and facilitate healing. Bone may also in grow into the cannulation and vent holes to assist in healing. One may appreciate that this one-step anchor system facilitates insertion and saves time during the medical procedure.

FIGS. 26A and 26B depict side and cross-sectional views, respectively, of another exemplary embodiment of an anchor 510a. As previously described in connection with similar embodiments, the anchor 510a may comprise a threaded portion 514a and a tip portion 512a. The anchor 510a may include a proximal surface 516a located a proximal end 518a and a distal surface 520a located at a distal end 522a. The anchor 510a may be cannulated, that is, the anchor 510a may have a bore 530a (see FIGS. 26B and 26D) which extends from the proximal surface 516a to the distal surface 520a. The threaded portion 514a may include a threaded portion body 515a having a thread 524a. The thread 524a may comprise crests, flanks, and roots, as previously described. The thread 524a may comprise a thread angle which can desirably be selected to facilitate insertion of the anchor 510a into bone or tissue. The thread 524a may extend continuously from the proximal surface 516a to the distal surface 520a, or some portion thereof. The threaded portion body 514a may comprise PEEK, metal, a combination thereof, or other materials.

The threaded portion 514a may include a suture opening 528a. Suture opening 528a may allow for insertion of a suture, tape, or thread to extend from the bore of the threaded portion body 515a, through the suture opening 528a, and to the outer surface of the anchor 510a. Suture opening 528a may accommodate one or more sutures, tapes, or threads. The suture opening 528a may comprise a length and a width, which may be selected to accommodate one or a plurality of sutures, tapes, or threads of desired size. The suture opening 528a may be located near the proximal end 518a of the anchor 510a. The suture opening 528a may be located some distance from the proximal surface 516a of the anchor 510a. Although a first suture opening 528a is depicted, it is understood that second, third and/or fourth suture openings could be located at various locations on the anchor 510a, including diametrically opposed and/or spaced apart from the first suture opening 528a. Any number of suture openings 528a may be provided in any number of locations about the axis of anchor 510a. The suture opening 528a may be located in the thread of the threaded portion 514a. The suture opening 528a may be located through the crest, flank, root, or any combination thereof of the thread. The threaded portion 514a may also include one or more holes 154a, which can extend through the threaded portion 514b and communicate with the bore 530a therein, such as to allow blood and/or other biologics (i.e., graft, etc.) to pass through the anchor as desired.

Referring to FIGS. 26C and 26D, the threaded portion 514a of anchor 510a is shown without an associated tip portion inserted therein (see FIGS. 7A through 7C for an another exemplary tip portion). The threaded portion is substantially the same as previously described with relation to FIGS. 1-6D, without the tip portion. For FIGS. 26C and 26D, it may be appreciated how a tip portion can be received by the threaded portion. Although the tip and threaded portions are depicted and described as two components, it will be understood that the anchor may be unitary, monolithic, or single piece anchors. As previously described, the threaded portion may comprise a threaded portion body. The threaded portion body may extend from a proximal surface to a threaded portion distal surface. The threaded portion may include a bore which extends from the proximal surface to the threaded portion distal surface.

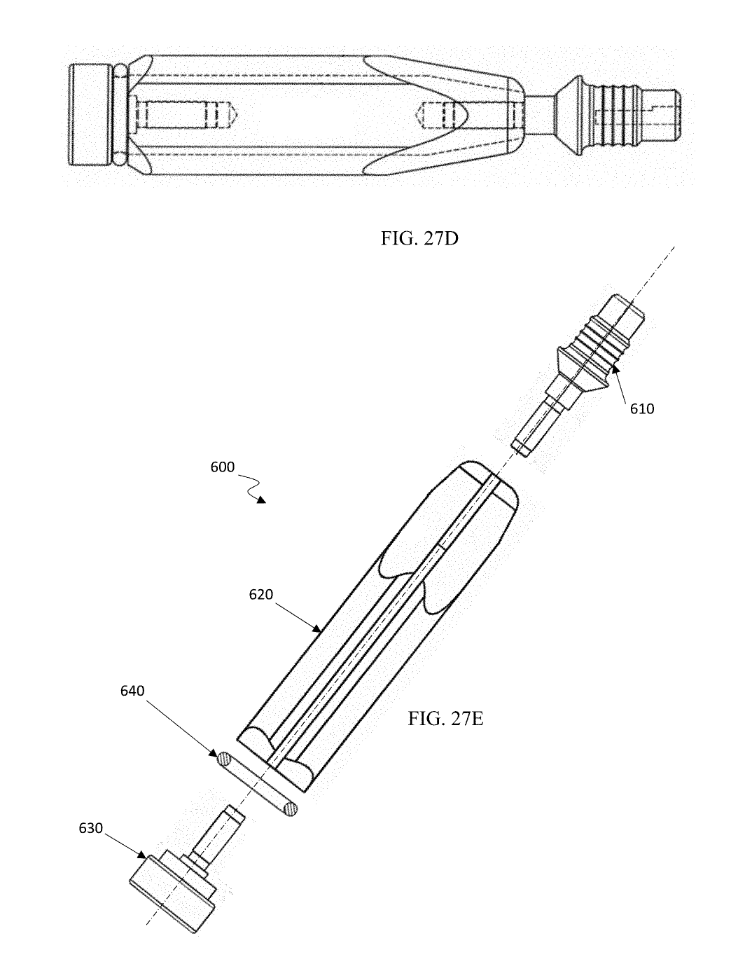

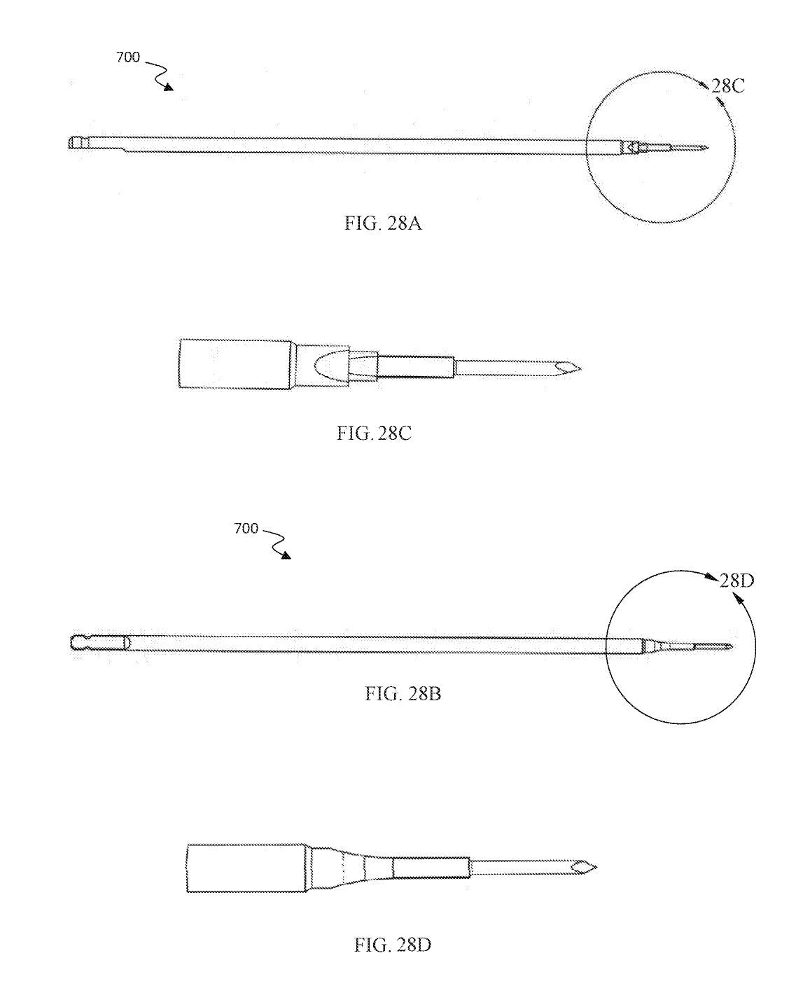

FIGS. 27A through 27E depict another exemplary embodiment of a modular handle 600 for use with various components of the present invention. In this embodiment, the handle 600 may include a quick-connect interface 610, such as the AO quick connect interface disclosed in U.S. Pat. No. 9,447,803B1, the disclosure of which is incorporated herein by reference. Desirably the interface (or other connection and/or “chuck” types well known in the art) will allow the handle 600 to accommodate a variety of surgical tools, including ratcheting adapters, torque limiters and/or multipliers, counter torque wrenches, awls, punches, trephines and/or the like. In the disclosed embodiment, the handle 600 can also accommodate a surgical awl 700 (see FIGS. 28A through 28D), which desirably accommodates the anchor of FIG. 26A and can be used for implantation of the anchor as describe herein.

In the disclosed embodiment, as best seen in FIG. 27E, the handle 600 includes a handle body 620, a proximal handle plug 630, an O-ring 640, and optionally a quick disconnect adapter 610. The handle also desirably includes a plurality of longitudinally extending external alleyways or channels 650, which can be sized and configured to accommodate sutures and/or suture tapes when an awl attachment 700 (see FIG. 28A) is connected to the handle 600. The incorporation of the O-ring 630 into the handle desirably allows the suture to not tangle when the anchor is being inserted into the bone.

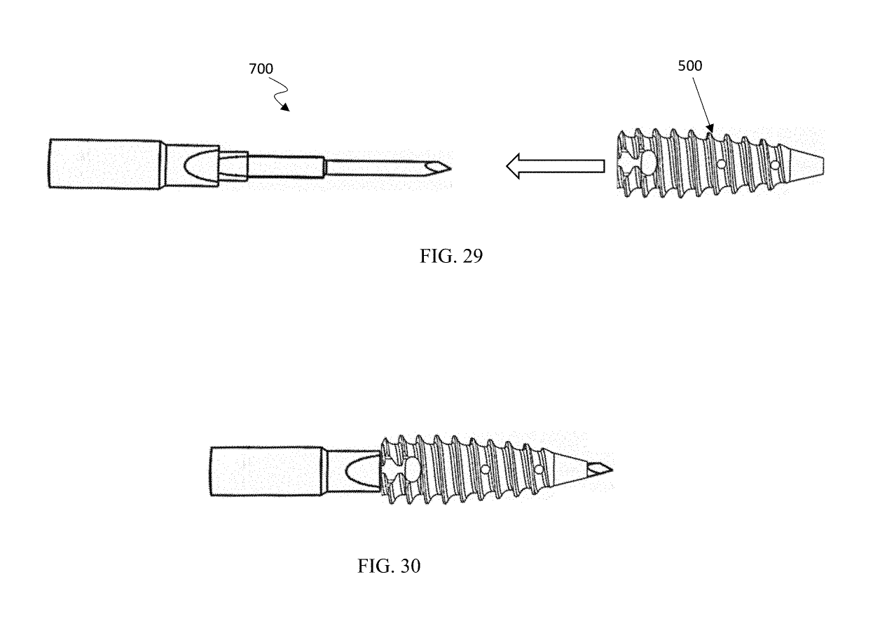

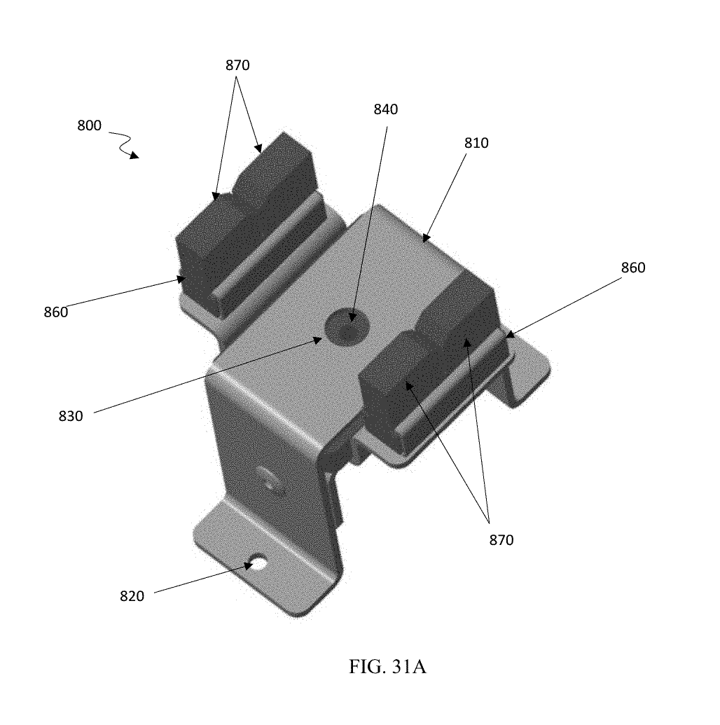

FIGS. 28A and 28B depict top and sides view of a modular awl 700 that can be utilized with various hand-operated components disclosed herein, and/or can also be utilized with powered surgical tools, if desired. FIGS. 28C and 28D depict expanded views of the anchor engagement tip of the awl 700, with FIGS. 29 and 30 depicting an exemplary engagement of the anchor engagement tip with an anchor 500a.

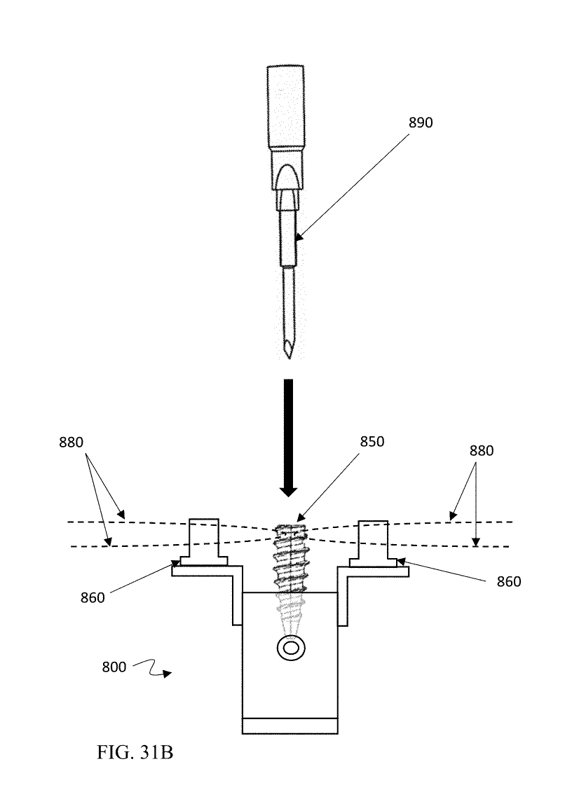

FIGS. 31A and 31B depict one exemplary embodiment of an anchor dock and holder 800 for use with the various anchor embodiments disclosed herein. The dock 800 includes a dock body 810 having fixation holes 820 for attachment within a surgical tray (not shown) or other location, such as the surgical back table in an operating room. The dock 800 further includes an anchor tip opening 830, which in various embodiments can be lined with a silicone or foam rubber insert 840 for accommodating and securing an anchor body 850 within the opening 830. The dock 800 further includes one or more suture retention clips 860 (with two such suture retention clips shown in the embodiment of FIG. 31A), which desirably include retention members 870 for engaging with and retaining sutures after they have been passed through suture openings (not shown) in the anchor body 850. During use, the anchor 850 will be inserted into the anchor tip opening 830, and then one or more sutures and/or suture tapes 880 can be inserted through the openings in the anchor body and the suture inserted into and/or retained within each suture retention clip 860. Once all of the suture tapes 880 have been inserted and retained in this manner, a surgical awl 890 can be inserted into the central bore of the anchor body, and the suture tapes can be attached to the handle (not shown) as previously described. The “loaded” anchor and associated surgical tool are now ready for use.

Desirably, the dock 800 will comprise sterilizeable materials that would allow the dock to be sterilized as part of an anchor implant kit and/or a surgical tool kit. Alternatively, the dock 800 could be supplied individually in a sterile condition and utilized in a single use and/or disposable manner.

The disclosed embodiments allow the disclosed anchors to be provided in a non-preloaded condition, if desired, such that the surgeon has the intraoperative flexibility to load whatever suture or tape the surgeon feels is best for the surgery into the anchor. There are many different surgeon preferences for tapes and/or sutures, as well as the potential need for different colors, sizes and/or styles of tape in a single surgery. The disclosed embodiments desirably allow an anchor to be utilized with any style, color and/or size of surgical suture and/or tape, which allows the surgeon to customize their anchor for whatever type of repair he or she needs.

Desirably, the suture openings on the anchor are sized and configured to accommodate three or more suture tapes within a single hole, which in various embodiments can allow 6 or more suture tapes to be utilized with a single anchor (i.e., where the anchor incorporates two suture holes). This is a significant improvement on many existing anchor designs, which may only accommodate a single tape in a single anchor hole and/or no more than two suture tapes to anchor (i.e., where the anchor incorporates two suture holes).

One may appreciate from the foregoing that a hybrid anchor may be provided that comprises a metal tip portion and a PEEK threaded portion. The awl may include a medial shaft having a metal tip. The metal tip may engage the bone and may be forced or screwed into the bone, either by hand or by power. The metal tip, which is removable from the anchor as previously described, in conjunction with the metal tip portion of the anchor and PEEK threaded portion of the anchor, may be screwed, either by handle or by power, into the bone to install the anchor into the bone. The modular shaft design and hybrid metal/PEEK anchor allow the insertion into the bone and also prevent the sutures or suture tapes from becoming tangled during insertion.

In one aspect of the invention, the tool including the cooperating awl and anchor as described in the various implementations described herein, can be utilized to install suture tape in a bone in a single step. This can be accomplished by passing a first suture tape along the threaded outer surface of the cannulated anchor, through the proximal suture opening of the cannulated anchor, and through the longitudinal bore of the cannulated anchor. A second suture tape may be threaded through a second suture opening and along a similar path as the first suture tape on an opposing side of the anchor. The awl's medial shaft is inserted through the longitudinal bore of the cannulated anchor such that a pointed tip of the medial shaft extends past a distal end of the cannulated anchor and such that the suture tape is located in an annular space between the medial shaft and an interior surface of the cannulated anchor. A hole may be punched in the bone with the pointed tip followed by the tip portion of the cannulated anchor impacting the bone. The bone hole may be enlarged by turning the awl such that the threaded outer surface of the cannulated anchor advances into and enlarges the hole. The awl may be removed from the cannulated anchor by pulling the handle of the awl to disengage the mating surfaces of the medial shaft and the cannulated anchor. The suture tape(s) may be secured to a tissue adjacent the bone. The awl can be reused to repeat the procedure with additional anchor(s) and suture tape(s).

As previously described, the anchors 10, 100, 1000 may comprise PEEK, however other materials, such as titanium may be contemplated. Alternatively, the anchors 10, 100, 1000 may have the threaded portion 14, 114, 1014 comprising a first material, such as PEEK and the tip portion 12, 112, 1012 comprising a second material, such as metal. When a metal tip portion is provided, the anchor may be facilitate insertion through hard or extremely hard bone. The metal may be any metal material known to those of skill in the art.

As may also be appreciated from the foregoing description, the suture openings located near the proximal end of the anchor are large enough that two suture tapes, one on each side of the anchor, may be installed. Each suture tape being separated may allow for larger fixation area after sutures are passed through the tissue or tendon.

The foregoing disclosure describes the advantages of the invention when used with threaded devices including sutures, such as the previously described anchors. Threaded devices without sutures, such as screws, may not experience the same dynamic loading problems which arise in threaded devices including sutures. For example, due to the extra weight and offset center that is present when sutures are included in an anchor and run longitudinally along an insertion tool, loading and forces present on threaded devices with sutures may be different that those present on threaded devices without sutures. Additionally, screws are often made of titanium, which is harder than bone. In use, a hole is predrilled into the bone and then the screw is inserted, either by power drill or hand screw, into the bone. The foregoing description is particularly beneficial to the dynamics of anchors including sutures and allows for an anchor to be inserted into the bone in a single step, either by hand or with a power drill.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1. A suture anchor comprising:
a threaded portion, the threaded portion including:
a body;
a suture opening in a side of the body, the suture opening configured to receive a suture tape;
a continuous thread on an outer surface of the body;
a bore extending from a body proximal surface to a body distal surface; and
a tip portion, the tip portion including an outer surface without the continuous thread and a bore extending from a tip portion proximal surface to a tip portion distal surface, the tip portion being tapered from a tapered portion proximal surface to the tip portion distal surface,
wherein the bore of the threaded portion aligns with the bore of the tip portion, thereby creating a continuous bore from the body proximal surface to the tip portion distal surface such that the suture anchor is open at the body proximal surface and open at the tip portion distal surface.

2. The suture anchor of claim 1, wherein the outer surface of the tip portion is smooth and the tip portion is frustoconical.

3. The suture anchor of claim 1, wherein the suture opening is located at a proximal end of the threaded portion.

4. The suture anchor of claim 1, wherein the continuous thread includes a crest, a flank, and a root, and wherein the suture opening extends through at least one of the crest, the flank, and the root.

5. The suture anchor of claim 1, further comprising the bore of the threaded portion having a first bore, a second bore, and a third bore; and
a vent opening in the body, the vent opening configured to allow flow of fluids through the suture anchor,
wherein the suture opening connects to the first bore and the first bore is located at a proximal end of the threaded portion, and
wherein a diameter of the third bore is substantially the same as a diameter of the bore of the tip portion.

6. The suture anchor of claim 1, wherein the bore of the threaded portion is multi-dimensional and wherein the multi-dimensional bore is configured to provide a gap between the suture anchor and a tool inserted through the suture anchor.

7. The suture anchor of claim 1, wherein the tip portion further comprises a non-tapered portion extending from the tip portion proximal surface to the tapered portion proximal surface and a tapered portion extending from the tapered portion proximal surface to the tip portion distal surface, and wherein the tapered portion proximal surface is configured to mate with the body distal surface.

8. The suture anchor of claim 1, wherein the continuous thread extends from the body proximal surface to the body distal surface.

9. The suture anchor of claim 1, wherein the threaded portion comprises polyether ether ketone and the tip portion comprises metal.

10. The suture anchor of claim 1, further comprising one or more ventilation openings.

11. A cannulated anchor system, comprising:
a suture anchor, the suture anchor including:
a body, the body including an outer surface and an inner surface defining a longitudinal bore;
a suture opening in a side of the body, the suture opening configured to receive one or more suture tapes and the opening located at a proximal end of the body; and
a medial shaft, the medial shaft having a pointed tip,
wherein the medial shaft is configured to extend through the longitudinal bore of the body of the suture anchor with the pointed tip extending past a distal end of the suture anchor, and wherein the cannulated anchor system is configured to allow one-step insertion of the suture anchor into a bone.

12. The cannulated anchor system of claim 11, further comprising a handle, the handle connected to the medial shaft, wherein the handle and the medial shaft are separate and one of the handle and the medial shaft is reusable.

13. The cannulated anchor system of claim 12, further comprising a metal tip on the suture anchor.

14. The cannulated anchor system of claim 11, wherein the body of the suture anchor further comprises a threaded portion and a tip portion, the tip portion comprising a smooth outer surface.

15. The cannulated anchor system of claim 14, wherein the threaded portion includes a continuous thread from a proximal surface of the thread portion to a distal surface of the threaded portion, and wherein a proximal surface of the tip portion is configured to mate with the distal surface of the threaded portion.

16. The cannulated anchor system of claim 11, further comprising an annular space between the medial shaft and the inner surface of the body, wherein the annular space is configured to allow passage of the one or more suture tapes.

17. The cannulated anchor system of claim 16, wherein the annular space is configured to allow passage of at least three stacked suture tapes.

18. A method of installing a suture tape in a bone in a single step, the method comprising:
providing a cannulated anchor having a threaded outer surface, a longitudinal bore, and a first opening extending through the threaded outer surface and located at a proximal end of the cannulated anchor;
passing a first suture tape along the threaded outer surface, through the first opening of the cannulated anchor, and through the longitudinal bore;
inserting a medial shaft through the longitudinal bore such that a pointed tip of the medial shaft extends past a distal end of the cannulated anchor and such that the first suture tape is located in an annular space between the medial shaft and an interior surface of the cannulated anchor;
punching a hole in the bone with the pointed tip;
impacting the bone with a tip portion of the cannulated anchor;
enlarging the hole by turning the medial shaft such that the threaded outer surface of the cannulated anchor advances into and enlarges the hole;
removing the medial shaft from the cannulated anchor; and
securing the first suture tape to a tissue adjacent the bone.

19. The method of claim 18, further comprising threading a second suture tape through a second opening and securing the second suture tape to the tissue.

20. The method of claim 18, further comprising reinserting the medial shaft in a second cannulated anchor for installing into another location in the bone.
